(19) United States
(12) Patent Application Publication
(10) Pub. No.: US 2008/0004398 A1
(43) Pub. Date: Jan. 3, 2008

(54) Bioactive Biomaterials for Controlled Delivery of Active Principles

(76) Inventors: Marie-Christine Durrieu, Villenave D'Ornon (FR); Damien Quemener, Castillon La Bataille (FR); Charles Baquey, Le Haillan (FR); Valerie Sabaut-Heroguez, Merignac (FR)

Correspondence Address: YOUNG & THOMPSON, 745 SOUTH 23RD STREET, 2ND FLOOR, ARLINGTON, VA, 22202, US

(21) Appl. No.: 11/630,513

(22) PCT Filed: Jun. 21, 2005
(86) PCT No.: PCT/FR05/01545

§ 371 Date: Feb. 20, 2007

(30) Foreign Application Priority Data
Jun. 21, 2004 (FR) 0406708

(51) Int. Cl. A61L 27/34; A61L 27/54

(52) U.S. Cl. 525/69; 528/392

(57) ABSTRACT

The invention relates to biomaterials comprising a carrier material to which surface spherical particles are covalently linked, wherein said spherical particles are formed by polymer chains containing approximately from 30 to 10000 monomer units derived from monocyclic polycyclic alkene polymerisation, are substituted by an R chain comprising ethylene polyoxide which is optionally covalently linked to said polymer units through a hydrolysable bridge and substituted by a reactive function engaged in a link with an active principle, said chain R being covalently linked to said monomer units. The use of the inventive biomaterials for preparing pharmaceutical and cosmetic compositions or surface coatings is also disclosed.

The present invention relates to bioactive biomaterials for the controlled delivery of active ingredients, as well as a method of synthesis thereof, and uses thereof.

The general aim of the present invention is to give the implantable devices the capacity to resist the development of different infectious and/or inflammatory processes which may follow their installation.

Nowadays, in order to alleviate these effects it is proposed to administer a medicament by the general route (A) as well as to administer antibiotics locally in bone surgery (B).

A—When a medicament is administered by the general route, it is distributed in the entire organism and its concentration at the intended site (namely a location which is capable of being the site of an infectious and/or inflammatory and/or neoplastic process) can only exceed the threshold of efficacy if the dose administered is sufficiently high at the risk of exposing the patient to toxic effects.

Substances pharmacologically administered by the oral or parenteral route frequently have a half-life which is too short and risks of general toxicity to achieve the desired local effect.

B—Since 1970 cements with antibiotics have been used in articular prosthetic surgery. In France there are 2 preparations on the market using either gentamycin or a combination of erythromycin and colimycin. It is also possible to prepare “cement with antibiotics”, particularly with vancomycin, in the operating theatre in non-standard conditions. The limiting factor of this method is the uncontrolled release (in terms of concentration and duration) of the active ingredient used. In fact, the kinetics of release of the active ingredient is not controlled since no device makes it possible to adjust its delivery and therefore to perpetuate its action over a predefined duration. Moreover, part of the active ingredient is not released because it is trapped too deep in the cement.

In order to remedy these drawbacks, systems for delivery of active ingredients, so-called “drug delivery systems (DDS)” have been developed. The principle of these drug delivery systems is to deliver pharmacologically active substances in situ, in a prolonged and regular manner, in a sufficient and non-toxic quantity.

Moreover, stimulable polymers, namely which are polymers sensitive to an external stimulus such a variation in pH or in temperature, have already been described which exhibit reactive functions obtained by encapsulation or adsorption of the active ingredients directly in the material or in beads which are themselves adsorbed or grafted on the material.

However, adsorption does not allow a controlled release of the active ingredient. As regards encapsulation when it can allow, on the other hand, a controlled release of the active ingredient, on the other hand, it proves incompatible with prolonged use and/or when the material is subjected to high stresses (flux, friction . . .).

Reactive polymer nanoparticles obtained by covalent grafting of active ingredients on the functionalised nanoparticle have also already been described. However, the synthesis of such nanoparticles takes place in two steps (synthesis of the latex, then reaction with the active ingredient) and therefore without direct control of the grafting (random number of functions introduced). Moreover, these nanoparticles do not further possess the active ingredient and anchoring sites allowing a release at a specific location of the active ingredient. These materials are most often intended for vectorisation or for immunological tests.

The present invention aims at proposing biomaterials which allow the controlled release, at the site of implantation of these biomaterials (over an adjustable period of time), of an active ingredient covalently fixed on the surface of this latter by chemical anchoring of spherical particles (particularly nanoparticles) functionalised by the active ingredient. A reaction of cleavage of the particle/active ingredient bond, actuated by the contact of the material with the physiological medium or by a modification of the pH, releases the bioactive molecule in its native form in a controlled manner. The concept may be extended to the local delivery of factors capable of regulating the relationship between an implant and the tissues surrounding it. The principle area of application is the biomedical field and more precisely the biomaterials used in vascular, endovascular (stent) and bone surgery.

Thus the present invention results from the demonstration of the fact that it is possible to fix to the surface of biomaterials spherical polymer particles which are bioactive and stimulable, that is to say sensitive to external stimuli such as a variation in pH or in temperature, exhibiting at their periphery reactive functions of the type of acid, amine, alcohol or acid chloride, these particles being obtained in one single step. The bonding of the active ingredient on the material is advantageously a bond which is hydrolysable under the effect of the pH and/or the temperature.

The invention relates to biomaterials comprising a support material which has covalently bonded on its support surface spherical particles having a diameter between 10 nm and 100 μm, said particles being formed by polymer chains containing about 30 to 10000 monomer units, identical or different, derived from the polymerisation of monocyclic alkenes in which the number of carbon atoms constituting the ring is approximately 4 to 12 or polycyclic alkenes in which the total number of carbon atoms constituting the rings is approximately 6 to 20, the said monomer units being such that:

at least approximately 0.5% of them are substituted by a chain R comprising an ethylene polyoxide of formula (A) optionally covalently bonded to the said monomer units via a hydrolysable bridge
—(CH₂—CH₂—O)ₙ—X (A)
wherein n represents an integer from approximately 50 to 340, especially from 70 to 200, and X represents an alkyl or alkoxy chain with about 1 to 10 carbon atoms, comprising a reactive function of the OH, halogen, NH₂, C(O)X₁ type, wherein X₁ represents a hydrogen atom, a halogen atom, an OR′ or NHR′ group in which R′ represents a hydrogen atom or a hydrocarbon chain with about 1 to 10 carbon atoms, substituted or unsubstituted, said reactive function being capable of bonding to a reactive function situated on said support material in order to ensure the covalent bonding between said material and said particles,

and at least approximately 0.5% of them are substituted by a chain R comprising an ethylene polyoxide of the aforementioned formula (A) wherein said reactive function is engaged in a bond with an active ingredient, or a biological molecule such as a protein, the said chains R being covalently bonded to the said monomers.

The invention relates more particularly to biomaterials as defined above, wherein the monomer units are derived from the polymerisation of monocyclic alkenes and are of the following formula (Z1)
═[CH—R₁—CH]═ (Z1)
wherein R₁ represents a hydrocarbon chain with 2 to 10 carbon atoms, saturated or unsaturated, said monomers being optionally substituted by a chain R, or directly by a group X, as defined above.

The invention relates more particularly to biomaterials as defined above, wherein the monocyclic alkenes from which the monomer units are derived are:

cyclobutene leading to a polymer comprising monomer units of formula (Z1a) below: 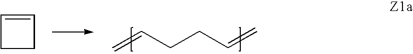
cyclopentene leading to a polymer comprising monomer units of formula (Z1b) below: 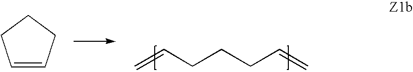
cyclopentadiene leading to a polymer comprising monomer units of formula (Z1c) below: 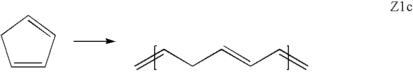
cyclohexene leading to a polymer comprising monomer units of formula (Z1d) below: 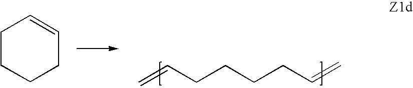
cyclohexadiene leading to a polymer comprising monomer units of formula (Z1e) below: 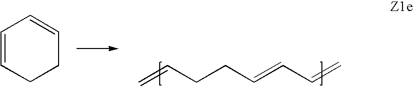
cycloheptene leading to a polymer comprising monomer units of formula (Z1f) below: 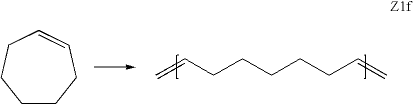
cyclooctene leading to a polymer comprising monomer units of formula (Z1h) below: 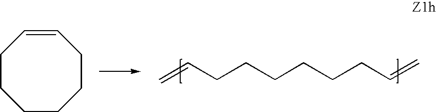
cyclooctapolyene, especially cycloocta-1,5-diene, leading to a polymer comprising monomer units of formula (Z1i) below: 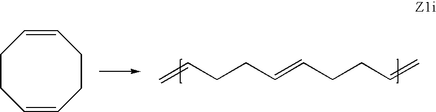
cyclononene leading to a polymer comprising monomer units of formula (Z1j) below: 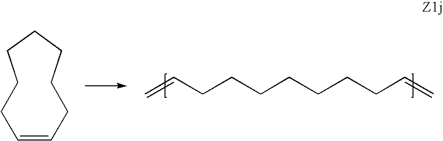
cyclononadiene leading to a polymer comprising monomer units of formula (Z1k) below: 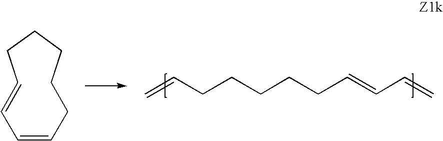
cyclodecene leading to a polymer comprising monomer units of formula (Z1l) below: 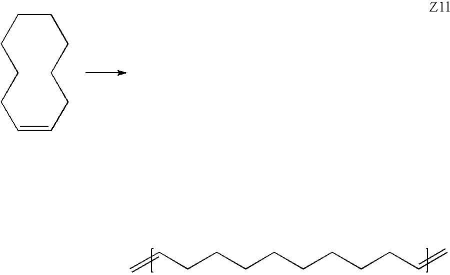
cyclodeca-1,5-diene leading to a polymer comprising monomer units of formula (Z1m) below: 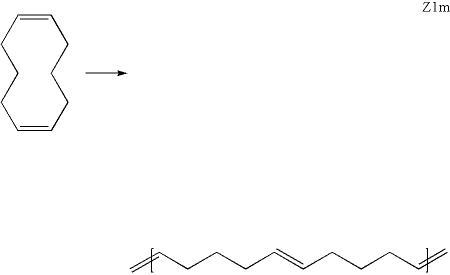
cyclododecene leading to a polymer comprising monomer units of formula (Z1n) below: 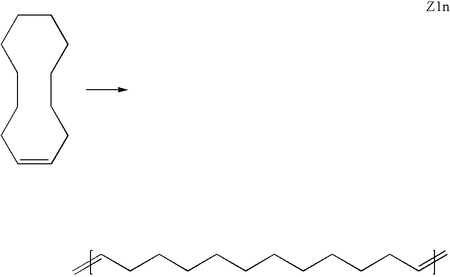
or also 2,3,4,5-tetrahydrooxepin-2-yl acetate, cyclopentadecene, paracyclophane, ferrocenophane.
The invention also relates to biomaterials as defined above, wherein the monomer units are derived from the polymerisation of polycyclic alkenes and are:

of formula (Z2) below:
═[CH—R₂—CH]═ (Z2)
wherein R₂ represents:

a ring of formula 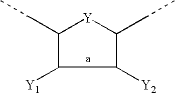
wherein:
Y represents —CH₂—, or a heteroatom, or a —CHR— group, or a —CHX— group, R and X being as defined above,
Y₁ and Y₂ independently of one another represent H, or a chain R, or a group X, as mentioned above, or form in association with the carbon atoms bearing them a ring with 4 to 8 carbon atoms, this ring being optionally substituted by a chain R or a group X as mentioned above,
a represents a single or double bond,

or a ring of formula 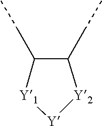
wherein:
Y′ represents —CH₂—, or a heteroatom, or a —CHR— group, or a —CHX— group, R and X being as defined above,
Y′₁ and Y′₂ independently of one another represent —CH₂—, or a —C(O) group, of a —COR group, or a —C—OX group, R and X being as defined above,
of formula (Z3) below: 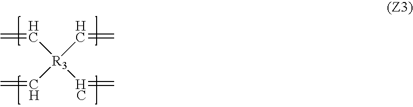
   wherein R₃ represents:

a ring of formula 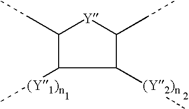
wherein:

n₁ and n₂, independently of one another, represent 0 or 1,

Y″ represents —CH₂—, or a —CHR— group, or a —CHX— group, R and X being as defined above,

Y″₁ and Y″₂ independently of one another represent a hydrocarbon chain with 0 to 10 carbon atoms,

or a ring of formula 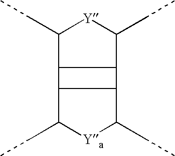
wherein Y″ and Y″a independently of one another represent —CH₂—, or a —CHR— group, or a CHX— group, R and X being as defined above,

or a ring of formula 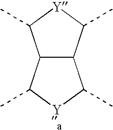
wherein Y″ and Y″a independently of one another represent —CH₂—, or a —CHR— group, or a —CHX— group, R and X being as defined above.

The invention relates more particularly to biomaterials as defined above, wherein the polycyclic alkenes from which the monomer units are derived are:

monomers containing a cyclobutene ring leading to a polymer comprising monomer units of formula (Z2a) below:

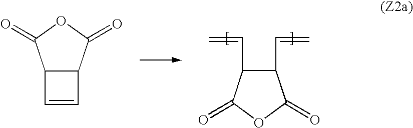

monomers containing a cyclopentene ring leading to a polymer comprising monomer units of formula (Z2b) below:

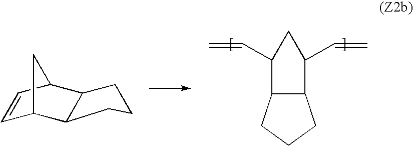

(bicyclo[2.2.1]hept-2-ene)norbornene leading to a polymer comprising monomer units of formula (Z2c) below:

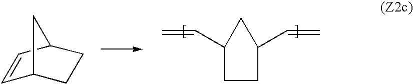

norbornadiene leading to a polymer comprising monomer units of formula (Z2d) below:

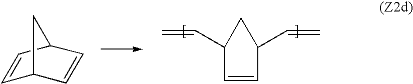

7-oxanorbornene leading to a polymer comprising monomer units of formula (Z2e) below:

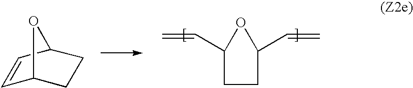

7-oxanorbornadiene leading to a polymer comprising monomer units of formula (Z2f) below:

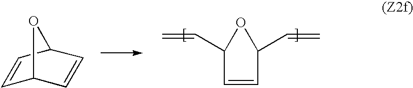

the dimer of norbornadiene leading to a polymer comprising monomer units of formula (Z3a) below:

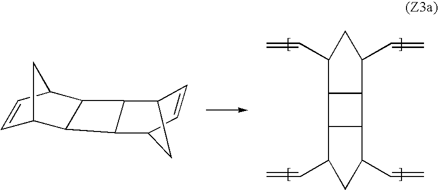

dicyclopentadiene leading to a polymer comprising monomer units of formula (Z3b) below:

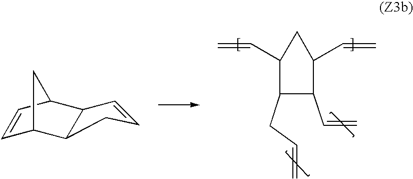

tetracyclododecadiene leading to a polymer comprising monomer units of formula (Z3c) below:

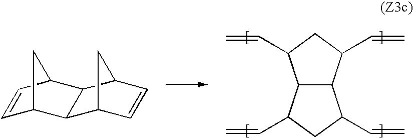

or bicyclo[5.1.0]oct-2-ene, bicyclo[6.1.0]non-4-ene.

The invention relates more specifically to preferred biomaterials as defined above, wherein the monocyclic or polycyclic alkenes from which the monomer units are derived are:

norbornene(bicyclo[2.2.1]hept-2-ene) leading to a polymer comprising monomer units of formula (Z2c),

tetracyclododecadiene leading to a polymer comprising monomer units of formula (Z3c),

dicyclopentadiene leading to a polymer comprising monomer units of formula (Z3b),

the dimer of norbornadiene leading to a polymer comprising monomer units of formula (Z3a),

cycloocta-1,5-diene leading to a polymer comprising monomer units of formula (Z1i).

The invention relates more specifically to biomaterials as defined above, wherein they comprise:

between about 0.5% up to 100% of monomer units substituted by a chain R as defined above, the said chain R being identical for these monomers, and comprising a reactive function capable of bonding to a reactive function situated on the said support material in order to ensure the covalent bond between the said material and the said particles,

and between about 0.5% and 99.5% of monomer units substituted by a chain R as defined above, the said chain R of these monomers being identical for these monomers, in which the said reactive function is engaged in a bond with an active ingredient, or a biological molecule such as a protein,

and/or between about 0.5% and 99.5% of monomer units directly substituted by a group X as defined above, this group X of these monomers being identical to or different from the group X of the chain R of the preceding monomers,

and/or between about 1% and 99.5% of unsubstituted monomer units,

the total of the percentages of the different monomers mentioned above being 100%.

The invention relates more particularly to biomaterials as defined above, wherein the chain or chains R substituting the monomers are represented by the formula
—CH₂—O—(CH₂—CH₂—O)ₙ—CH₂—CH₂—O—X
wherein n is as defined above, and X represents H, —CH₂—COOH, —CH₂—COCl, —CH₂—COY, wherein Y depicts an active ingredient, or a biological molecule such as a protein.

The invention also relates to biomaterials as defined above, wherein the chain or chains comprise an ethylene polyoxide of formula (A) bonded covalently to the said monomer units by a hydrolysable bridge.

Such materials are especially advantageous insofar as they permit a controlled release of the active ingredients which are stable or unstable in vivo. According to this strategy the release of the active ingredient trapped inside the particle, and therefore isolated from the external medium, and bonded covalently to the particle, is effected by a first step of destabilisation of the said particles by breaking the bonds between the monomer units and the chains R via an external stimulus (such as pH, hyperthermia . . .), which involves salting out of the stabilising chains R. In a second reaction time the resulting chains R or Z1, which are or are not functionalised by the active ingredient, undergo hydrolysis reactions and release the active ingredient.

The materials according to the invention are materials which have bonded on their surface spherical particles which are stimulable, namely sensitive to an external stimulus such as a variation in pH or in temperature, which then allows the release of the active ingredients trapped inside these particles.

The hydrolysable bridges mentioned above are preferably chosen from amongst the chain formations having approximately 1 to 10 units of ε-caprolactone, or —OC(O)—, —C(O)OC(O)—, C(O)—NH— . . . functions.

In this connection the invention relates more particularly to biomaterials as defined above, wherein the chain or chains R comprising an ethylene polyoxide of formula (A) bonded covalently to a hydrolysable bridge chosen from amongst the chain formations having approximately 1 to 10 units of ε-caprolactone are represented by the formula
—CH₂—(O—CO—(CH₂)₅)ₜ—O—CO—(CH₂)₅—O—CO—(CH₂)₂—CO—O—(CH₂—CH₂—O)ₙ—(CH₂)₂—O—X
wherein t represents an integer between 1 and 10, and X represents H, —CH₂—COOH, —CH₂—COCl or —CH₂—COY, Y representing an active ingredient, or a biological molecule such as a protein.

The biomaterials according to the invention as defined above are advantageously wherein wherein the support material is chosen from:

metals, such as titanium,

metal alloys, in particular alloys with or without shape memory such as Ni—Ti alloys,

polymers, such as polyethylene terephthalate (PET), polytetrafluoroethylene (PTFE), polyvinylidine fluoride (PVDF), polyether etherketone (PEEK),

copolymers, such as the copolymer ethylene vinyl acetate (EVA), the copolymer vinylidene fluoride-hexafluoropropylene P(VDF-HFP), poly(lactic acid)-co-poly(glycolic acid) (PLA-PGA),

ceramics, such as hydroxyapatites, or compounds of hydroxyapatites and tricalcium phosphate in varied proportions, in particular in the proportions 50/50.

The invention also relates to biomaterials as defined above, wherein the reactive function situated on the support material in order to ensure the covalent bond between the said material and the said particles by reacting with the reactive function of these latter is of the type of the OH, halogen, NH₂, C(O)X′₁, wherein X′₁ represents a hydrogen atom, a halogen atom, an OR″ or NHR″ group, wherein R″ represents a hydrogen atom or a hydrocarbon chain with approximately 1 to 10 carbon atoms, substituted or unsubstituted, in order to form a bond of the —O—C(O)—, —NH—C(O)—, —C(O)—NH—, —C(O)0— or —C(OC)₂ type with the reactive function of said particles.

The invention relates more particularly to biomaterials as defined above, wherein the reactive function of the support material is situated on an alkyl chain having about 1 to 10 carbon atoms grafted on said material, substituted or unsubstituted, and optionally comprising one or several heteroatoms, in particular O, and Si, in said chain.

The invention relates more particularly to biomaterials as defined above, wherein:

the reactive function of the material is an NH₂ function situated on an aminopropyltriethoxysilane molecule grafted on the material according to the following formulae:

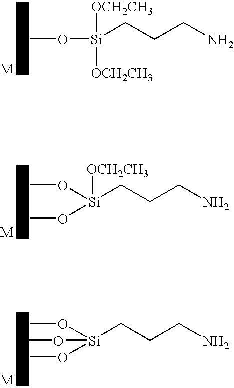

wherein M represents a metal oxide or a ceramic such as hydroxyapatite or any other polymer having OH sites on its surface (naturally or due to prefunctionalisation),

the reactive function of the material is an NH₂ function situated on a surface prefunctionalised by acrylic acid which is coupled to a bifunctional spacer arm such as bNH₂PEG (O,O′-bis-(2-aminopropyl)-polyethylene glycol 500 (this prefunctionalisation is described in the article Nucl. Instr. And Meth. in Phys. Res. B 151 1999 255-262).

The invention also relates to biomaterials as defined above, wherein the active ingredient is chosen from molecules used in therapy, cosmetics, perfumery, or for surface coatings in order to rendering them uncolonisable by different types of microorganisms (algae, fungi, bacteria . . .) such as paints and antifouling coatings.

The invention relates more specifically to biomaterials as defined above, wherein the active ingredient is a medicament used in therapy chosen in particular from those in the following therapeutic categories: antibiotics, antiinflammatories, antimitotics, hormones, growth factors.

The invention also relates to the use of biomaterials as defined above for the preparation of implantable medical devices, in particular in the form of implants, prostheses, stents or cements, in particular in vascular, endovascular or bone surgery.

The invention also relates to implants, prostheses, vascular stents or cements as well as any pharmaceutical composition comprising biomaterials as defined above.

The invention relates more particularly to biomaterials as defined above, wherein the biological molecule is chosen from the proteins capable of bonding to an intracellular or extracellular biological target, or to antibodies or to any other specific ligand.

The invention relates more particularly to biomaterials as defined above, wherein the biological molecule is chosen from amongst the following proteins: avidine, albumin, growth factors such as VEGF.

The invention also relates to pharmaceutical compositions comprising biomaterials as defined above, in which the different group(s) X contain a medicinally active ingredient, optionally in association with a pharmaceutically acceptable carrier.

The invention also relates to cosmetic compositions comprising biomaterials as defined above, in which the different group(s) X contain(s) an active ingredient used in cosmetics, optionally in association with an appropriate carrier, in particular for an application in the form of emulsions, creams.

The invention also relates to compositions for surface coatings comprising spherical particles as defined above, in which the different group(s) X contain(s) an active ingredient used for the surface coatings, optionally in association with an appropriate carrier.

The invention also relates to a method of preparation of biomaterials as defined above, wherein it comprises:

a step of polymerisation of a monocyclic or polycyclic alkene as defined above substituted by a chain R as defined above, optionally in the presence of:
one or several monocyclic or polycyclic alkenes as defined above, identical to or different from the foregoing, and substituted by a chain R as defined above, said chain R being different from that substituting the aforementioned monocyclic or polycyclic alkene,
and/or one or several monocyclic or polycyclic alkenes as defined above, identical to or different from the foregoing, and substituted by a group X as defined above, this group X being identical to or different from the group X of the chain R of the preceding alkenes,

and/or one or several monocyclic or polycyclic alkenes as defined above, identical to or different from the foregoing, said alkenes being unsubstituted,

said polymerisation being carried out while stirring in the presence of a transition metal complex as initiator of the reaction chosen in particular from those in groups IV or VI or VII or VIII, such as ruthenium, osmium, molybdenum, tungsten, iridium, titanium, in a polar or apolar medium, particularly with the aid of the following ruthenium-based complexes: RuCl₃, RuCl₂(PCy₃)₂CHPh . . . .

and a step of fixing said spherical particles obtained in the previous step on a support material as defined above by placing the said particles in the presence of the said material, this latter having been optionally functionalised with a reactive function as defined above capable of ensuring the covalent bond between the said material and the said particles by reacting with the reactive function of the said particles.

The invention will now be illustrated in support with the following detailed description of obtaining biomaterials according to the invention and the physicochemical characteristics of the particles.

The synthesis of the spherical particles is carried out in one step and allows the kinetics of release of active molecules to be easily modified as a function of the envisaged application. Furthermore, the fact that this object can be grafted covalently on the material gives it excellent properties of mechanical stability and ensures that they are stable over time.

The use of the spherical particles makes it possible not only to increase the specific surface area of the material in order to guarantee a sufficient concentration of bioactive molecules but also to introduce several chemical functions or active ingredients easily on the surface of the biomaterial.

Schematically, the production of the proposed device can be broken down into three distinct steps:
1—The functionalisation of the biomaterial
2—The synthesis of the bioactive nanoparticles
3—The fixing of the nanoparticles on the biomaterial
   1—The Functionalisation of the Biomaterial

In terms of materials, the development of a bioactive prosthesis necessitates control of the interfaces between materials and molecules or between materials and biomolecules. Grafting is a technique which allows one or several molecules chosen for their specific properties to be fixed by covalent bonding to the surface of any type of material. All of the treatment is carried out under controlled atmosphere, temperature and pressure, which enables perfect control of the grafting conditions. The technique employed consists of a modification of the functionality at the surface of the biomaterial in order to render it more reactive.

By way of illustration, the experimental conditions used in the case of grafting of a molecule of aminopropyltriethoxysilane (APTES) on hydroxyapatite (HA) (diagram 1) are set out below. 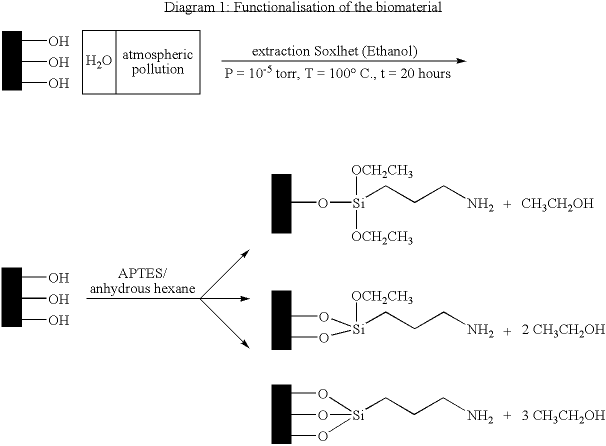
a) Preparation of the Surface

The HA is washed with the aid of a Soxlhet extractor (with ethanol) for 24 hours.

b) Modification of the Surface

The modification of the surface was carried out in a dry chamber devoid of air in order to avoid contamination of the surface by water and carbon compounds originating from the surrounding atmosphere and in order to ensure the reproducibility and the stability of the molecular layer. The strategy for immobilisation of the ligand (Diagram 1) involves the grafting of an amino-functional organosilane (APTES) on the hydroxyapatite surface (HA).

Experimentally, the modification of the HA surface was carried out using the following procedure, also shown in FIG. 1.

1. The HA was degassed at 100° C. in vacuo (10-5) for 20 hours (surface A).

2. The silanisation of the HA surface was carried out by immersing the substrate in a solution of APTES (1×10⁻² M) in anhydrous hexane under an Ar atmosphere for 2 hours whilst stirring.

3. The sample were washed under an AR atmosphere by 3 rinsings whilst stirring and sonication for 30 minutes (the two steps were carried out using anhydrous hexane).

4. The samples were degassed at 70° C. in vacuo (10⁻⁵ torr) for 4 hours (surface B).

c) Characterisation of the Surface

X-ray photoelectronic spectroscopy (XPS) was applied to the control of the reactions at each step of the procedure. The XPS spectra were recorded with the aid of a CG 220i-XL Escalab spectrometer on the HA substrates at each step of the grating of the RGD peptides. The power of the non-monochromatic MgKα source was 200 W with a studied zone of approximately 250 microns. An electron gun was used to compensate for the charges. The acquisition of high-resolution spectra was effected at constant energy flows of 20 eV. The adjustment was then carried out with the aid of software supplied by VG Scientific, each spectrum having as reference a carbon pollution at 284.8 eV. The bonding energy values (BE) are given as ±0.2 eV.

2—Synthesis of the Nanoparticles

The synthesis of the nanoparticles is carried out by copolymerisation in a disperse medium of vinyl monomers (cyclo-olefins) with macromonomers α,ω-functionalised by a polymerisable entity and by a reactive function and/or an active ingredient (medicaments, organic molecules . . .). Examples of this synthesis are detailed below.

A) Synthesis of Macromonomers of Formulae A and B Below

The macromonomers (A and B) are poly(ethylene oxide) oligomers with a molar mass ( M̅ₙ) of 7000 g/mol. They are derived from a “live” anionic polymerisation which allows control of the length and the functionality of the chains. They are functionalised at one of their ends by a norbornenyl unit, an entity chosen for its high reactivity in polymerisation by metathesis and, at the other end by a reactive function of the type of alcohol, acid, amine . . . (A), or by the active ingredient (indomethacin) (B) via a cleavable bridge (acid anhydride, ester, amide, . . .).

### 1. α-norbornenyl-ω-carboxylic acid-poly(ethylene oxide); formula A

Chemical Formula: 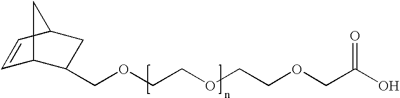
with n between 50 and 340 as a function of the requirements of the envisaged application.
Reference: NB-POE-COOH. 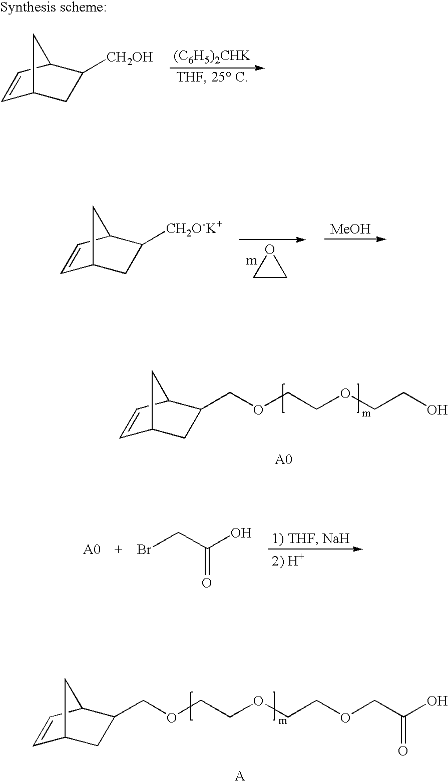
Procedure for Synthesis:

5-norbornene-2-methanol (0.5 mL) in solution in tetrahydrofuran (THF) (200 mL) is first of all deprotonated by the addition of a molar equivalent of diphenylmethyl potassium. The resulting radical will then initiate the polymerisation of ethylene oxide (28 mL) in a “live” manner (48 h) until the destruction of the active centres by the addition of methanol (1 mL). The alcohol function of the poly(ethylene oxide) obtained (A0) will then be transformed into an acid function by deprotonation of A0 (10 g) with NaH (0.17 g) in solution in THF (15 mL), followed by the addition of bromoacetic acid (0.42 g). After washing of the product with hydrochloric acid (18 mL, 1M) then precipitation in ether, the macromonomer A is obtained in a pure form.

### 2. α-norbornenyl-ω-indomethacin-poly(ethylene oxide); formula B

Chemical Formula: 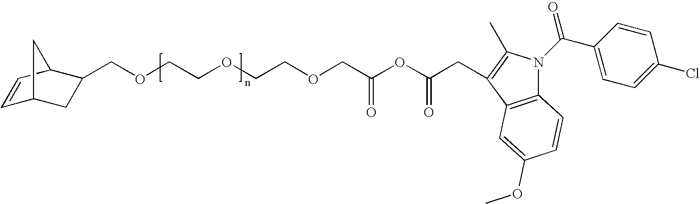
where n is between 70 and 200 as a function of the requirements of the envisaged application.
Reference: NB-POE-CO(O)-IND. 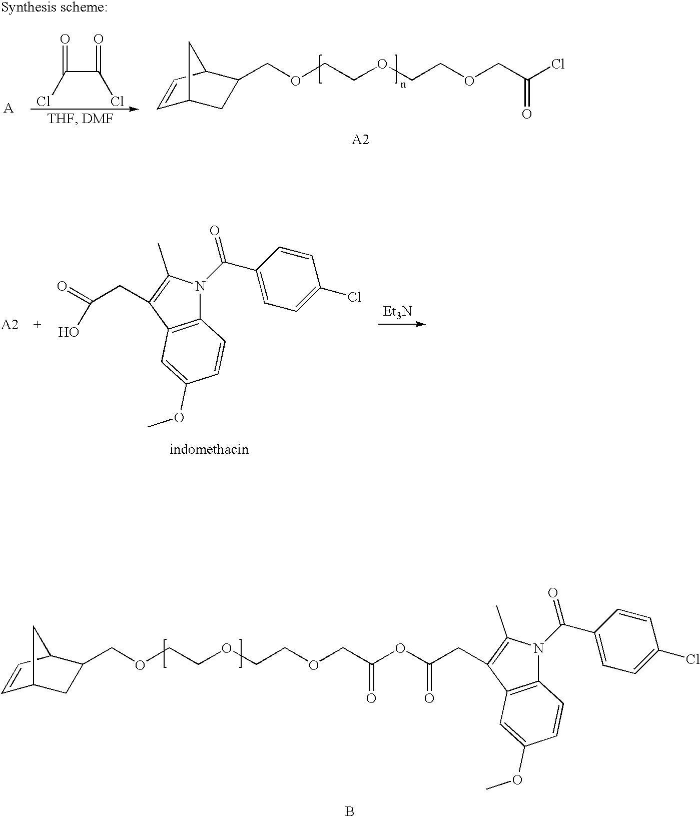
Procedure for Synthesis:

The acid function of NB-POE-COOH (A) is transformed into an acid chloride (A2) by reaction of A (5.2 g) on oxalyl chloride (0.08 mL) in THF (25 mL) in the presence of a catalytic quantity of dimethylformamide for 24 h. Indomethacin (0.6 g) as well as triethylamine (0.24 mL) are then added to the solution of A2 and left while stirring for 15 h. After precipitation in ether, the macromonomer B is obtained.

a-3. Indomethacin Derivative of Norbornene

The monomer used in the preceding reactions is norbornene (NBH) or norbornene functionalised (NBD) by the active ingredient. This latter is then introduced via a hydrolysable bridge of the type of ester, anhydride, amide . . . . The synthesis of norbornene functionalised by indomethacin is described below.
Chemical Formula: 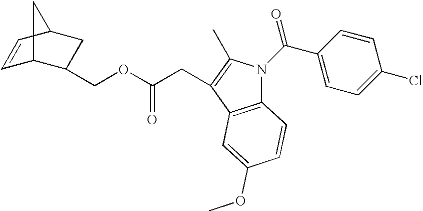
Reference: NBD. 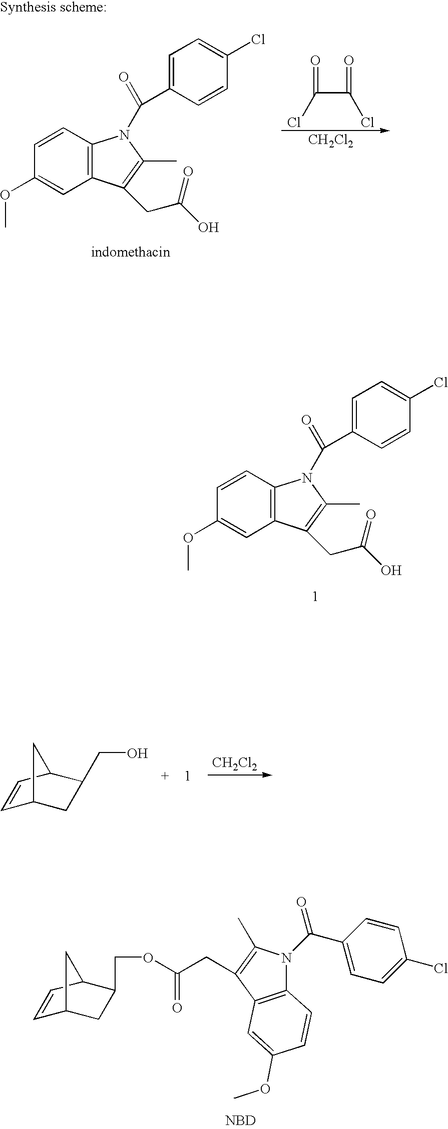
Procedure for Synthesis:
Synthesis of the Monomer NBD

During a typical reaction, oxalyl chloride (0.87 mL) is added to indomethacin (1.1 g) in solution in dichloromethane (20 mL). After 2 hours of reaction and elimination of the unreacted oxalyl chloride, the compound 1 obtained is then added to a solution of 5-norbornene-2-methanol (0.36 mL) in dichloromethane (20 mL) in the presence of triethylamine (0.84 mL) and left while stirring for 15 hours at 45° C. After purification by extraction, the monomer NBD is obtained (p>95%).

b. Synthesis of Particles

The particles according to the invention are obtained by copolymerisation in a dispersed medium (emulsion, mini-emulsion and micro-emulsion, dispersion, suspension) of vinyl monomers (cyclo-olefins) with macromonomers α,ω-functionalised by a polymerisable entity and a reactive function or an active ingredient (medicaments, organic molecules . . .). The polymerisation is initiated by transition metals and can be carried out in an aqueous or organic medium (dichloromethane/ethanol). Macromonomers play the part of stabiliser and functionalising agent. In the capacity of stabilisers they make it possible during the formation of the polymer in the reaction medium to disperse it in the form of spherical nanoparticles. From the purely steric point of view the stabilisation is insensitive to any variation in pH of the medium. Moreover, the functionalisation of latex by means of a macromonomer improves the availability of the reactive functions on the surface of the latex and preserves the reactivity thereof.

The initiator of the polymerisation is a ruthenium-based complex which is stable in a polar medium: RuCl₃, RuCl₂(PCy₃)₂CHPh and homologues thereof. Latex synthesised in these conditions will consist of polyalkenamer chains bearing poly(ethylene oxide) grafts, which will serve to stabilise the particles.

The particles obtained are stable in an aqueous and/or organic medium. Their size is between a few nanometres and a few micrometres as a function of the method of polymerisation used (dispersion, suspension, mini-emulsion . . .). The nanoparticles are spherical with very good isometry. 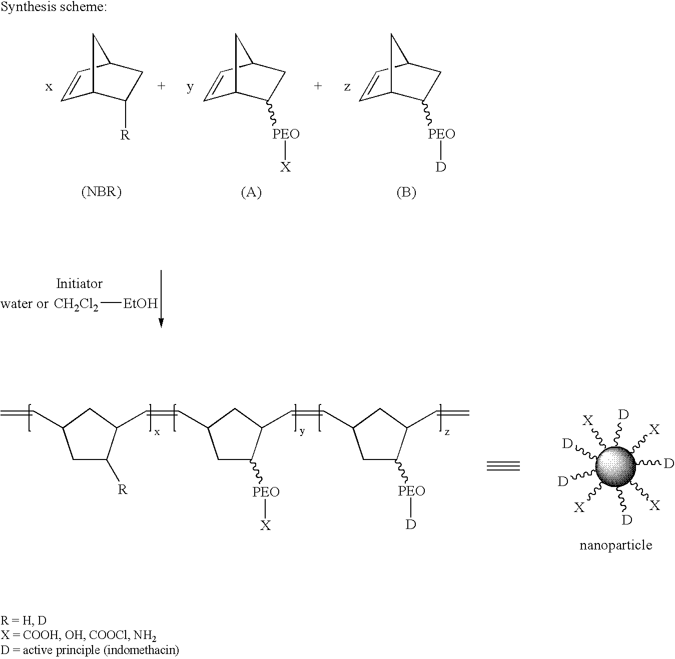
Procedure for Synthesis:

The macromonomers A and B are copolymerised in the presence of a monomer (NBH and/or NBD). In a typical reaction 0.8 g of monomer and 1 g of macromonomer (0.2 g of A and 0.8 g of B) previously dissolved in 14 ml of a dichloromethane/ethanol mixture (35%/65%) are added under a nitrogen atmosphere and with vigorous stirring to 10 ml of dichloromethane/ethanol (50%/50%) containing 20 mg of initiator. The duration of the polymerisation is one hour. The totally homogeneous starting medium becomes increasingly cloudy as the polymerisation takes place. Monitoring of the polymerisations by gas chromatography has revealed total conversions of monomers in less than one minute. The incorporation of the macromonomers A and B into the latex is total.

c. Variant for the Transport of Sensitive Active Ingredient

The latex is prepared as previously by copolymerisation between a cyclo-olefin (norbornene) which does or does not carry an active ingredient (indomethacin) and the stabilising polymer (NB-PCL-POE-OMe). This latter, which is or is not functionalised by a reactive function of the acid, acid chloride, alcohol, amine type (same function as previously), has a hydrolysable bridge, particularly units of ε-caprolactone (PCL) between the polymerisable function and the ethylene polyoxide chain according to the following scheme:

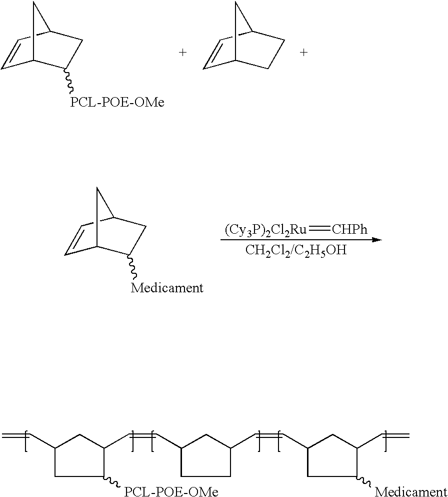

According to this process the release of the active ingredient trapped inside the particle and bonded covalently thereto (FIG. 13) necessitates a first step of destabilisation of the latex. This can be achieved via an external stimulus (pH, hyperthermia . . .) by salting out of the stabilising chains.

In a second reaction time the resulting linear chains of polyalkenamers functionalised by the active ingredient undergo hydrolysis reactions and release the active ingredient (FIG. 14).

c-1) Procedure for Synthesis of the Copolymer poly(caprolactone-β-ethylene glycol)-α-norbornene-ω-methyl ether NB-PCL-POE-OMe.

### Preparation of poly(caprolactone)α-norbornenyl (NB-Pcapro)

Triethyl aluminium (1.3×10⁻² moles) is added drop by drop to a solution of 2-hydroxymethyl-5-norbornene (1.3×10⁻² moles) in toluene (100 mL) cooled to −80° C. After a progressive return to ambient temperature the reaction is continued for 2.5 hours. Caprolactone (3.9 mole) is then added to the reaction medium with vigorous stirring. After 18 hours of reaction, 50 mL of hydrochloric acid (0.1 N) are added. After washing until neutral poly(ε-caprolactone)α-norbornenyl is precipitated cold in heptane then filtered on frit No. 4. The traces of heptane will be eliminated by heating (40° C.) in vacuo for 10 hours. The polymer obtained is then freeze-dried three times with dioxan as solvent.

### Preparation of poly(ethylene glycol)-α-carboxylic acid-ω-methyl ether

Solubilise 3.89×10⁻³ moles of succinic anhydride and 4.10×10⁻³ moles of triethylamine in 45 mL of anhydrous acetone. Whilst stirring, add drop by drop a solution of poly(ethylene glycol)monomethyl ether (6×10⁻⁴ moles) in 15 mL of anhydrous CH₂Cl₂. After 16 hours of reaction, add 1 mL of methanol. After concentration in a rotary evaporator, precipitate the polymer in ethyl ether. Recommence the steps of dissolution/precipitation two further times. Place the polymer in a dynamic vacuum for 10 hours to eliminate all traces of solvent.

### Preparation of the copolymer poly(caprolactone-b-ethylene glycol)-α-norbornene-ω-methyl ether (NB-Pcapro-PEG-OMe)

Solubilise 4×10⁻⁴ moles of poly(ethylene glycol)-α-carboxylic acid-ω-methyl ether in 40 mL of anhydrous CH₂Cl₂. Add oxalyl chloride (8×10⁻⁴ moles) to this solution cooled to 5° C. After 15 hours of reaction, remove the excess of unreacted oxalyl chloride as well as the CH₂Cl₂ under reduced pressure. The yellow residue obtained is then redissolved in 40 mL of dichloromethane. After having added triethylamine (4.3×10⁻⁴ moles), add α-norbornenyl poly(caprolactone). After concentration in a rotary evaporator, precipitate the polymer in ethyl ether. Recommence the steps of dissolution/precipitation two further times. Place the polymer under reduced pressure for 10 hours to eliminate all traces of solvent.

The synthesis of α-norbornenyl poly(caprolactone) is effected according to the following scheme: 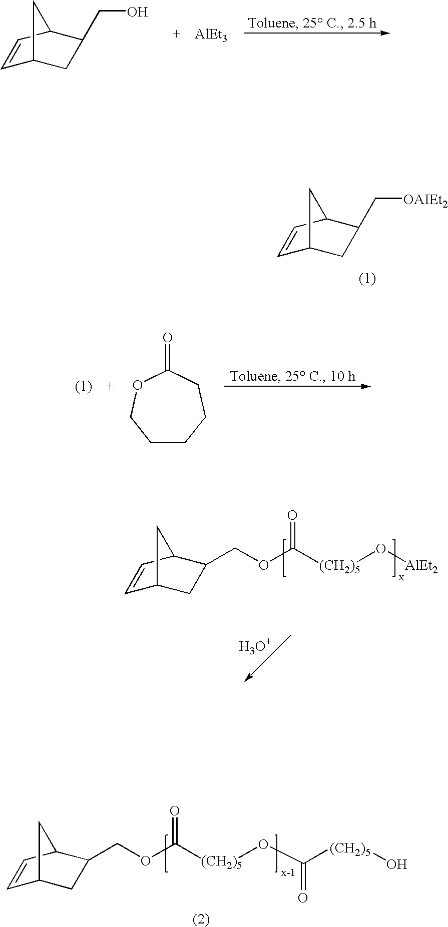
The synthesis of poly(ε-caprolactone-b-ethylene glycol)-α-norbornene-ω-methyl ether is effected according to the following scheme: 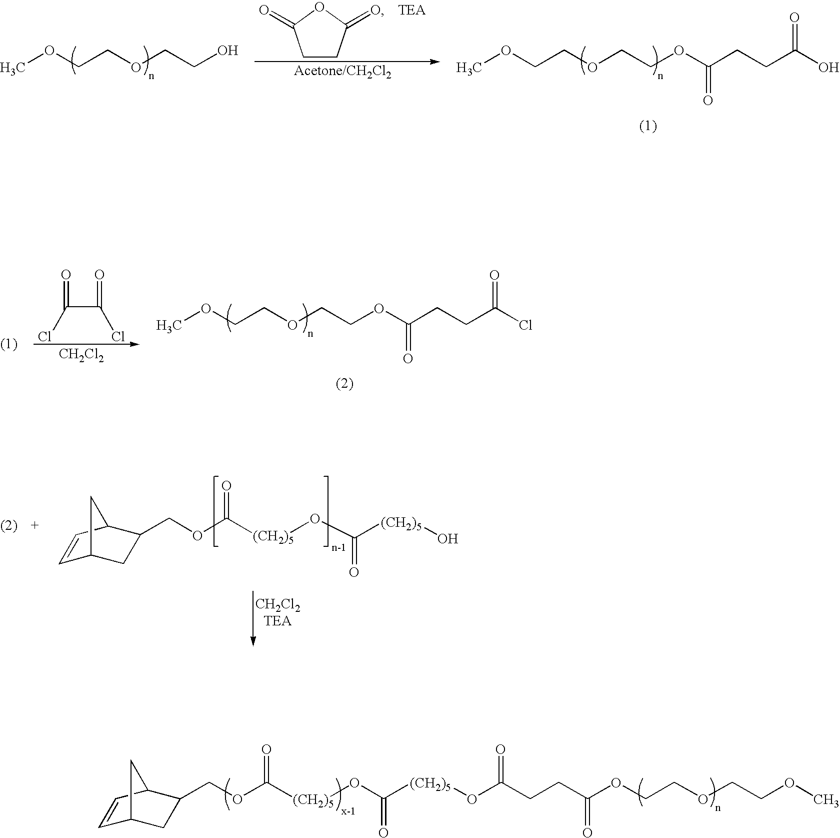
2) Release of the Active Ingredient

Once the particle was synthesised we verified by UV-visible spectrometry the possibility of releasing the medicament by simple lowering of the pH. The results obtained allowed confirmation of a progressive and controlled release of indomethacin. Moreover, the application of a pH equal to 3 revealed that more than 85% thereof could be salted out in 48 hours.

3—Fixing of the Nanoparticles on the Biomaterial

The covalent fixing of the particles on the material is carried out by condensation of two antagonistic reactive functions of which one is located on the material and the other on the particles. Mention may be made by way of example of the pairings acid/amine, acid/alcohol, acid/chloride, alcohol/acid chloride . . . .

In the case of interest to us here this reaction is effected between a material having reactive functions (in this case amines) and nanoparticles functionalised not only by bioactive molecules (in our example by indomethacin) and anchoring sites (in this case acids). The material is then said to be bioactive, that is to say that it possesses a biological activity (diagram 2). In our example this activity is stimulated by the release of the active ingredient in its native form when the material is in contact with the physiological medium or by a modification of the pH. For this a cleavable bond is introduced between the nanoparticle and the active ingredient 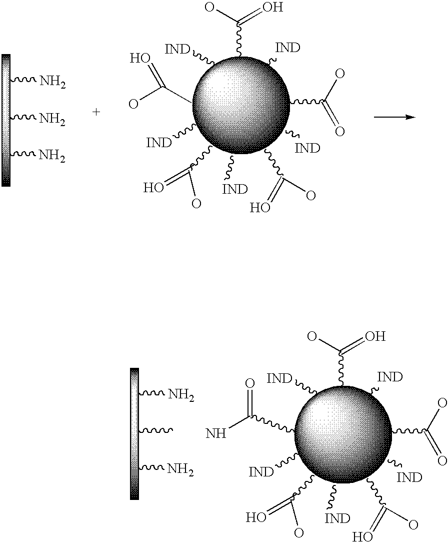
Diagram 2: Fixing of the Bioactive Nanoparticles on the Material

During a typical reaction 3×10⁻⁴ mol of hydroxybenzotriazole (HOBT) are added to a solution of nanoparticles (3.66×10⁻⁵ mol of acid functions) in 2 mL of dimethylformamide. After complete solubilisation of the HOBT the material is next introduced then left whilst stirring for 15 minutes at ambient temperature. 2.5×10⁻⁴ mole of 1-(3-dimethylaminopropyl)-3-ethylcarbodiimide is then added to the reaction medium which is maintained for 12 hours whilst stirring. The material functionalised by bioactive particles is then purified by successive washings then dried.

The topography of the hydroxyapatite materials has been studied by scanning electron microscopy (SEM) (FIGS. 15-17; enlargement: 20 000; FIGS. 18-19; enlargement: 5 000). The presence of nanoparticles has been clearly demonstrated by comparison of the topologies of the materials before (images in FIGS. 15, 16) and after (image in FIG. 17) the grafting reaction.

Moreover, the stability of the chemical anchoring of the nanoparticles was verified: no nanoparticle was released following an extraction with Soxlhet with ethanol for 12 hours at 80° C. (image in FIG. 18). The same treatment, applied to a material having simply physisorbed particles leads to the total extraction of the nanoparticles (image in FIG. 19).

Biofunctionalisation Procedure

XPS was used to follow each step of the reaction because it can supply information concerning chemical bonds and atomic concentrations. Table 1 gives modifications of the atomic proportions on the upper surfaces.

TABLE 1
Atomic composition by XPS in percentage of HA at each step
of the treatment

| | Ca | P | C | O | Si | N | N/Si |
|---|---|---|---|---|---|---|---|
| HA | 13.3 | 7.4 | 30.0 | 49.3 | — | — | — |
| HA + APTES | 7.0 | 5.5 | 45.5 | 34.0 | 4.0 | 4.0 | 1 |

FIGS. 20 and 21 show successively the spectra Cl and N1 obtained after each step of the treatment. The bonding energies (BE) mentioned are comparable with the BE found in the literature (Table 2). In the following discussion only the characterisation in XPS of the peptide cyclo-DfKRG is presented, and the procedure for grafting of GRGDSPC leads to similar results.

TABLE 2
Bonding energies (eV) assigned to specific functional groups
containing nitrogen, carbon and silicon according to the
data in the literature.

| | C1s | Si2p | N1s |
|---|---|---|---|
| SiO3C | 283.9 | 102.7 [1] | |
| CHx | 284.8 [2] | | |
| C—CO | 285.3 [3] [2] | | |
| C—NH2, C—O | 285.9-286.1 [2] | | 398.9 [4] |
| N—C═O | 286.4 [2] | | 399.7-400.8 [3] |
| Imide, Maleimide, | 287.2-288 [2] | | 399.7-400.7 [5, 6] |
| guanidine | | | |
| COOH | 288.5 [2] | | |

The principal elements in the hydroxyapatite surfaces are Ca, P, C and O. The expected theoretical ratio Ca:P is approximately 1.7. The experimental ratios obtained are successively 1.8, 1.3, for HA and HA+APTES respectively. After grafting of the surface, the detections of Ca and P are more difficult because the electrons must succeed in passing through the grafted layer.

The XPS analysis of HA treated by APTES confirms that silicon and nitrogen are detected in addition to Ca, P, O and C which are usually found on the surfaces of HA. Our experimental measures (Table 2) lead to a N:Ai ratio close to the theoretically expected ratio (4.0:4.0). In addition to the CHx bonds visible at 284.8 eV (FIG. 20b), the contributions of the carbon with oxide compounds of smaller size (at 285.4 eV) may correspond to residual ethoxy groups belonging to silane molecules. By comparison with the “A” spectrum of C1 (FIG. 20a), a new contribution is visible at 283.9 V. This latter peak is attributed to SiO3C groups (Table 2) in accordance with the peak appearing at 102.5 eV in the Si2p spectrum. At the same time the spectrum of N1 (FIG. 3a) reveals two components: one with low energy, characteristic of C—NH₂ groups (398.9 eV) and two others (at 401.7 eV and 400.2 eV) which may be attributed to the nitrogen involved in the oxidic environments. This latter contribution may be due to certain interactions between the terminal amine group and the oxygen group close to the surface. Based on all these observations it is evident that the —CH₂—CH₂—NH₂ chains are grafted well on the surface.

4—Study of the Release of the Active Ingredient

After grafting of the nanoparticle on the material the release of the active ingredient (in this case indomethacin) was achieved by contact of the material with the physiological medium. The results obtained made it possible to confirm the controlled salting out of the active ingredient without alteration of the surface of the material.

5—Cytocompatibility of the Extracts

The possible toxicity of a material with respect to cells may be researched by studying the effect caused by the extract of this material. These effects make it possible to demonstrate the toxic effect of distillable substances or soluble products of salting out.

a) Obtaining Extracts

According to the standard, extracts were produced by adhering to a ratio of the apparent surface area of the immersed part of the sample to the volume of the extraction medium between 3 and 6 cm²/ml. We chose to fix this ratio at 5 cm²/ml.

The extraction medium remains at the discretion of the experimenter: culture medium with or without serum, NaCl 9% solution or any other appropriate solution. We chose the culture medium.

The extraction is carried out in borosilicate glass tubes in order to avoid any interaction. The duration of this extraction is 120 hours in an incubator at 37° C. At the end of this extraction period the fragments of material are withdrawn and the liquid obtained corresponds to the extracts which will be used in the course of the tests.

We used the extract in pure (undiluted) or diluted form using the culture medium as diluent in order to obtain dilutions of 50% (v/v), 10% (v/v) and 1% (v/v). As solution of phenol (64 g/l in the culture medium) is inserted as “positive control”, that is to say capable of inducing a cytotoxic response in a reproducible manner.

b) Seeding of the Cells and Bringing Together of the Extracts and the Solutions

For the biocompatibility tests the cells are placed in 96-well culture plates (Nunc) which allow reading on a spectrophotometer (Laboratoire Dynatech, Saint-Cloud, France). The seeding density is 6000 cells/cm² for human osteoprogenitor cells. In 72 hours the cell mat has reached subconfluence, enabling the tests to be carried out.

c) Carrying Out of the Tests

These are colorimetric tests making it possible to demonstrate a cellular metabolic activity or simply the cell viability. The measurement of the intensity of the stained reaction with the aid of the spectrophotometer allows a quantitative evaluation.

c-1) Neutral Red Test

c-1-1) Principle

This test was developed by Parish and Mullbacher in 1983 in order to determine the cell viability. Neutral red is a vital stain which is fixed by electrostatic bonding to the anionic sites of the lysosomial membranes in the live cells. An alteration of this membrane causes a reduction in the fixing of the stain. The intensity of the stained reaction enables evaluation of the number of live cells after incubation in the presence of a toxic agent.

c-1-2) Protocol

The culture plates are withdrawn from the incubator after 24 hours of contact between the extraction liquid or the solutions and the cells, each well is rinsed at least twice with the aid of 0.2 ml of phosphate buffer. A 0.4% solution (v/v) of neutral red (Sigma) in the culture medium (100 μl/well) is distributed in each of the wells. After 3 hours' incubation at 37° C. the neutral red solution is removed and the extraction of the stain is carried out by the addition of 100 μl/well of a 1% solution (v/v) in water of acetic acid in 50% (v/v) of ethanol.

The plates are agitated for five minutes. In each of the wells a coloration of variable intensity is obtained of which the absorbency is measured with a spectrometer at the wavelength 540 nm. The coloration extends from colourless for colorations involving 100% toxicity to a more or less red colour for the control and the extracts which are not very toxic.

c-2) MTT Test

c-2-1) Principle

This test was introduced by Mosmann in 1983 in order to determine the cellular metabolic activity. MTT or 3-(4,5-dimethaziol-2yl)-2,5-diphenyl tetrazolium)bromide is a yellow-coloured tetrazolium salt in aqueous solution at pH neutral. It is metabolised by the mitochondrial dehydrogenase succinate of the live cells in blue formazan crystals. The quantity of formazan generated by the cells, after incubation in the presence of a toxic agent, gives an indication of the number and the metabolic activity of the live cells.

c-2-2) Protocol

The culture plates are withdrawn from the incubator after 24 hours' contact between the extraction liquid of the solutions and the cells, each well is rinsed at least twice with the aid of 0.2 ml of phosphate buffer. A solution of MTT (0.125 ml at 1 g/ml prepared in a Hanks buffer containing 1 g/l of glucose) is distributed in each well. The plates are replaced in the incubator for 3 hours in order that the expected enzymatic reaction should occur. After elimination of the supernatant, the formazan crystals formed are solubilised by the addition of 0.1 ml/well of DMSO (dimethyl sulphoxide, Sigma). The solubilisation of the crystals is instantaneous, but the coloration thereof is only stable for an hour. The plates are therefore rapidly read in a spectrophotometer at the wavelength of 540 nm, which makes it possible to obtain an absorbency value per well. The coloration extends from colourless for the concentrations involving 100% toxicity to a very deep purplish for the control and the extracts which are not very toxic.

### BIBLIOGRAPHY

[1] G. Josefsson, L. Lindberg, B. Wilander, “Systemic antibiotics and gentamicin-containing bone cement in the propylaxis of postoperative infections in total hip arthroplasty”, Clin. Orthop. 1981; 159: 194-200.

[2] A. D. Hanssen, D. R. Osmon, C. L. Nelson, “Prevention of deep periprosthetic joint infection”, J. Bone Joint Surg. 1996; 78-A: 458-471.

[3] C. P. Duncan, B. A. Masri, “The role of antibiotic-loaded cement in the treatment of an infection after hip replacement”, J. Bone Surg. 1994; 76-1: 1742-1751.

[4] D. Neut, H. van de Belt, J. R. van Norn, H. C. can der Mei, H. J. Busscher, “Residual gentamicin-release from antibiotic-loaded polymethylmethacrylate beads after 5 years of implantation”, Niomaterials 2003; 24: 1829-1831.

### LEGENDS ON THE DRAWINGS

FIG. 1: ¹H NMR spectrum of the macromonomer of formula A.

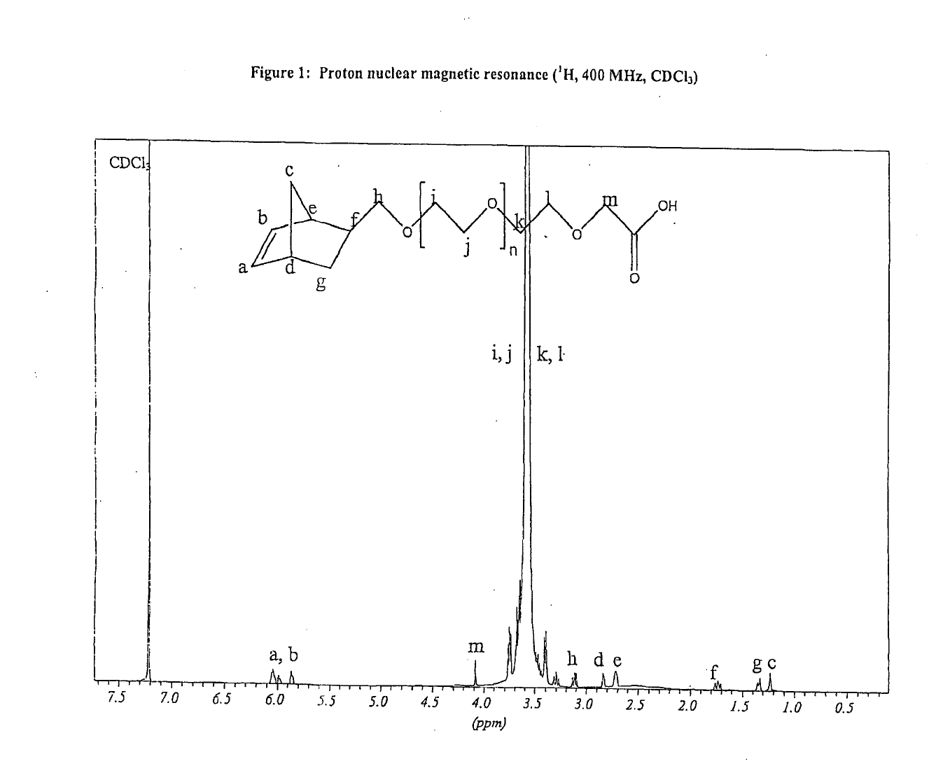

FIG. 2: ¹³C NMR spectrum of the macromonomer of formula A.

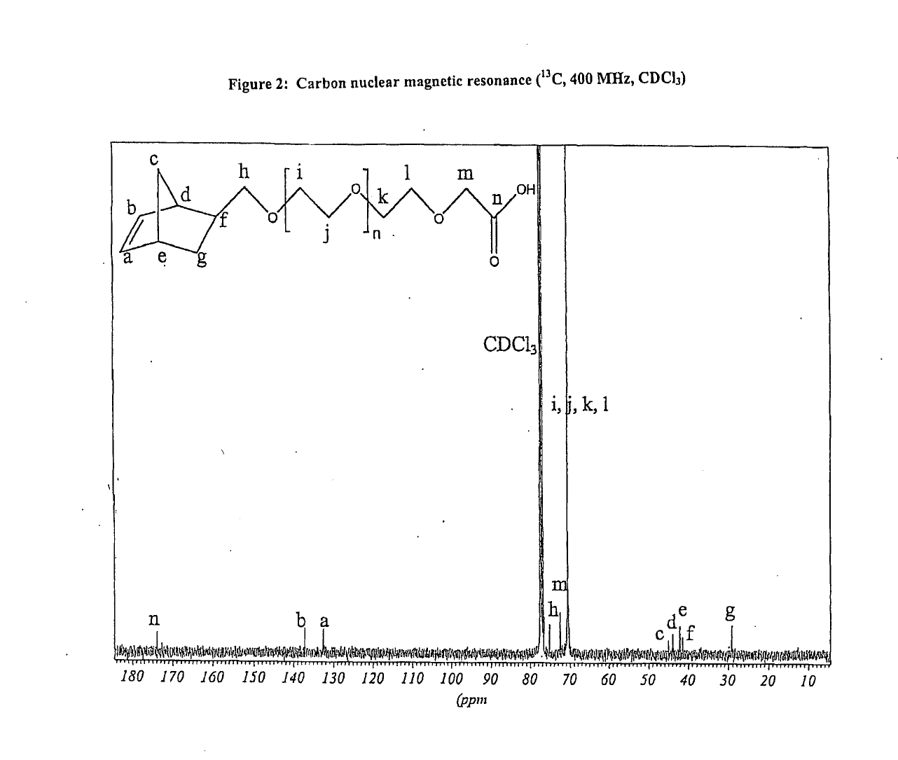

FIG. 3: Steric exclusion chromatography of the macromonomer of formula A in THF.

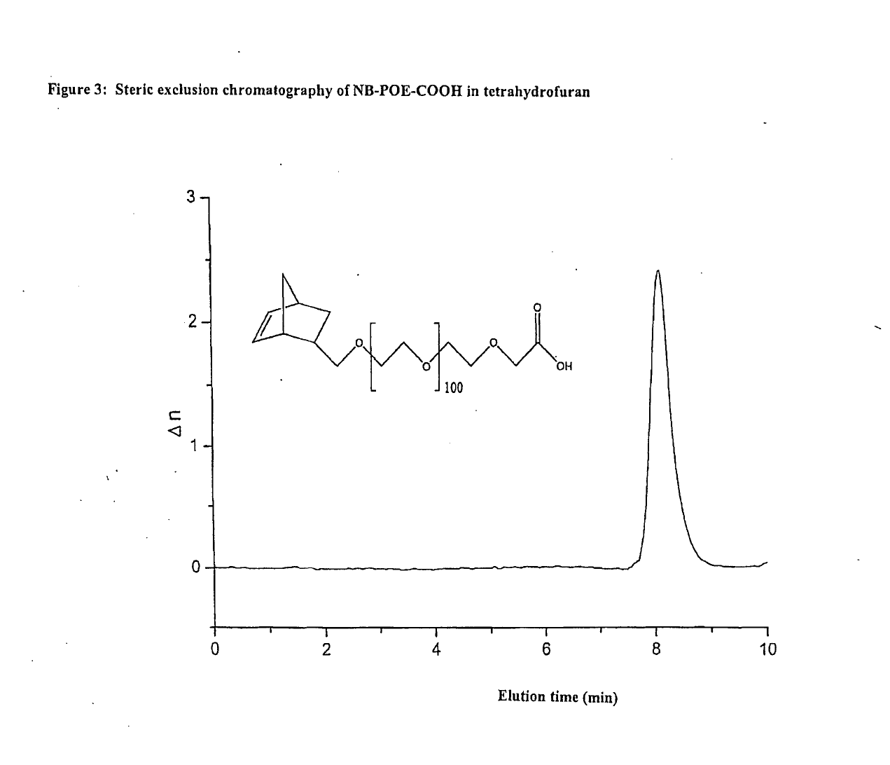

FIG. 4: ¹H NMR spectrum of the macromonomer of formula B.

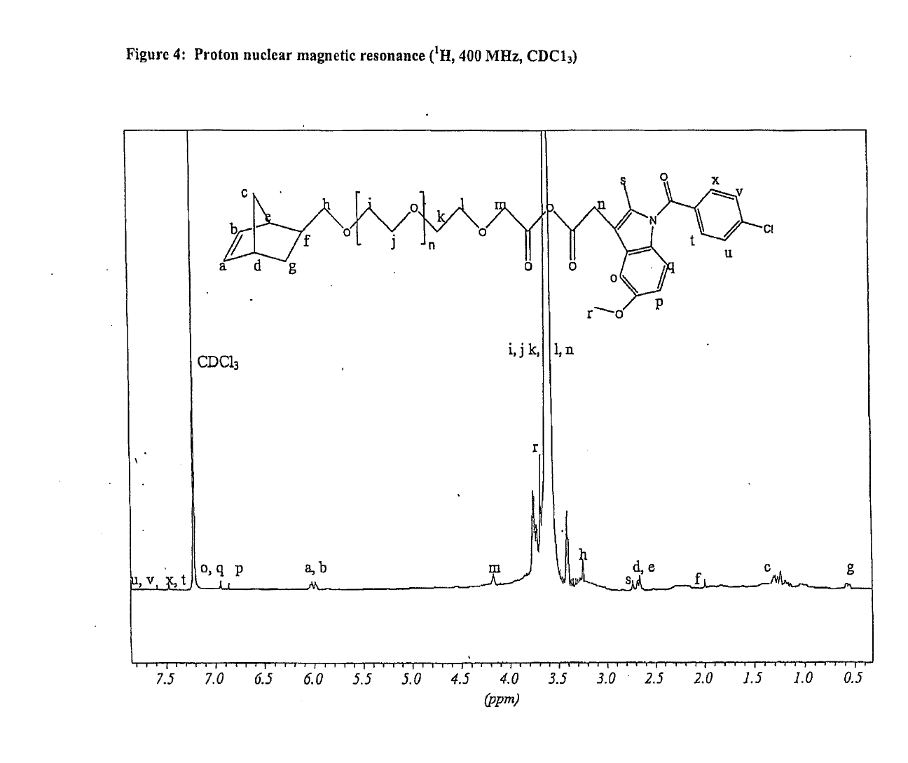

FIG. 5: Steric exclusion chromatography of the macromonomer of formula B in THF.

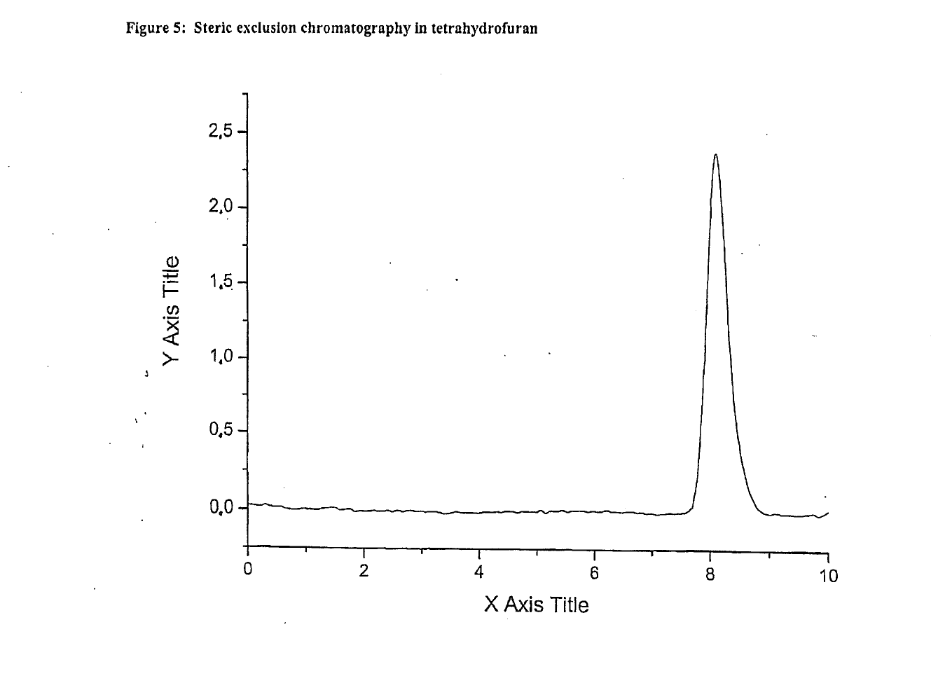

FIG. 6: ¹H NMR spectrum of the compound NBD.

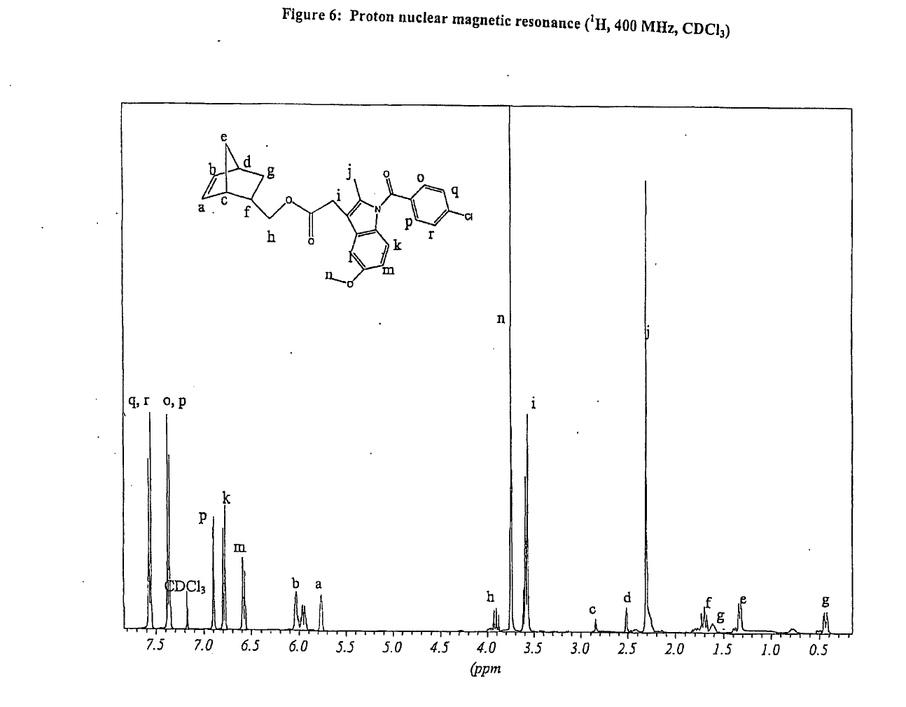

FIG. 7: ¹³C NMR spectrum of the compound NBD.

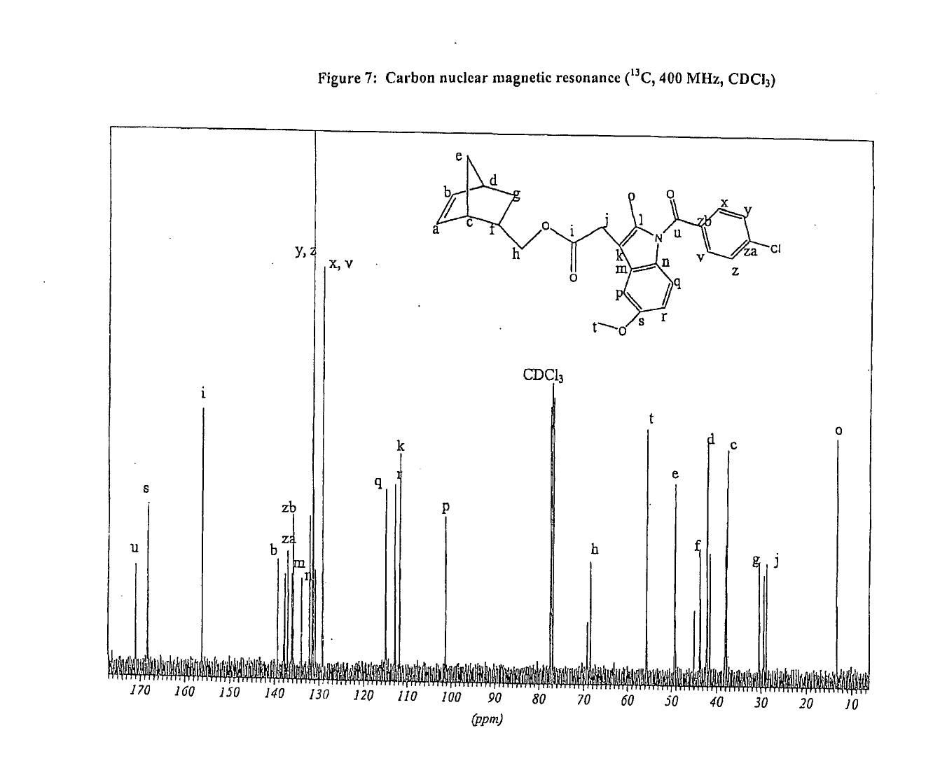

FIG. 8: Study of the conversion to NB and to NB-POE-CO(O)-IND during the polymerisation reaction. Evolution of the conversion to norbornene (♦, NB) and of the macromonomer (•, NB-POE-CO(O)-IND) as a function of time.

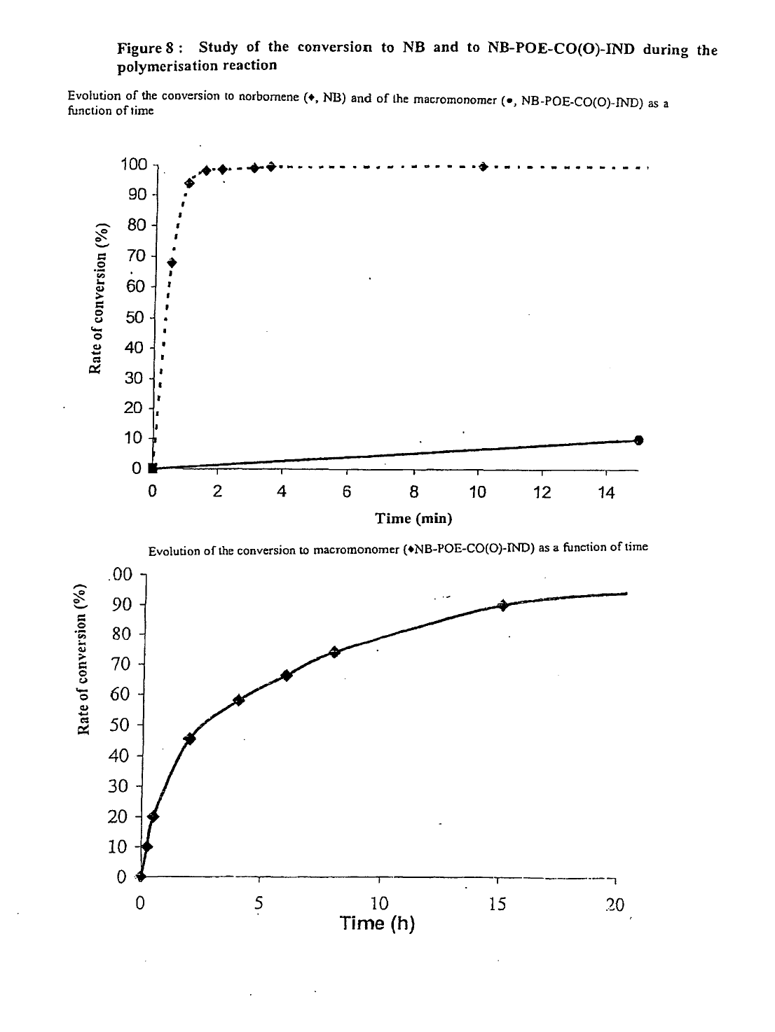

FIG. 9: Scanning electron microscope image of spherical particles obtained by copolymerisation of the macromonomers A and B in the presence of the monomers NBH and/or NBD.

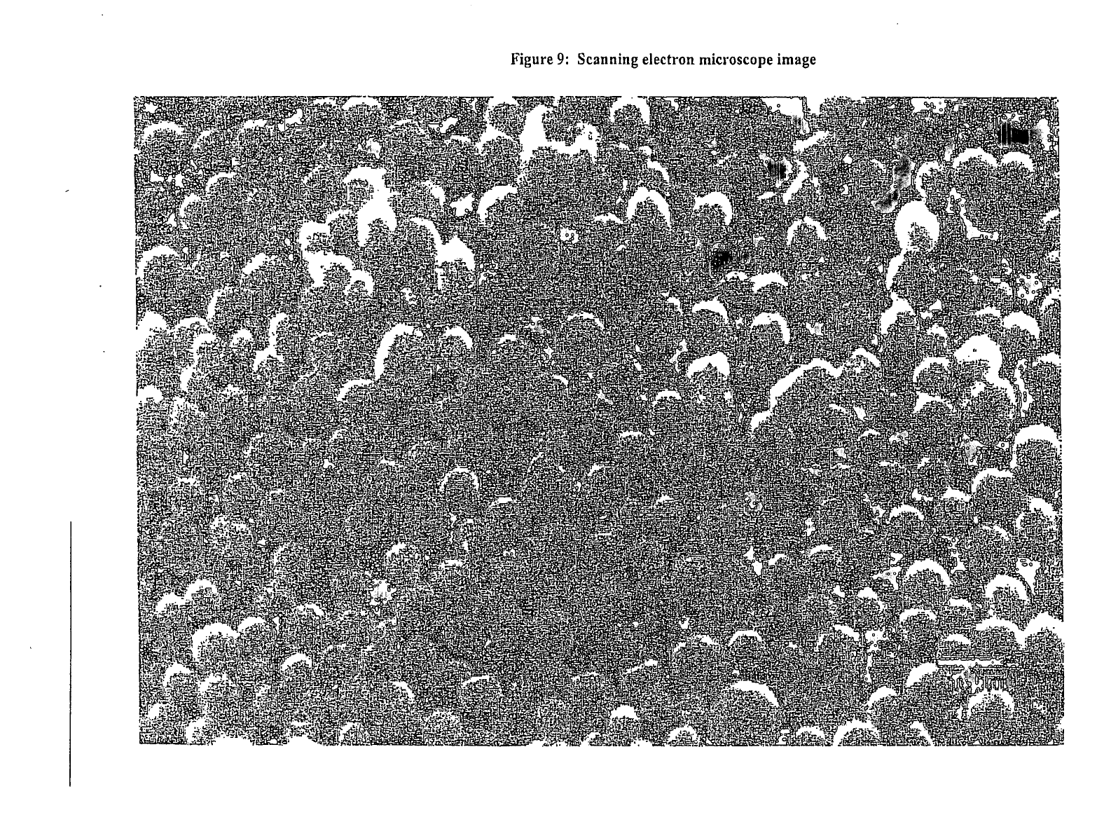

FIG. 10: Transmission electron microscope image of spherical particles obtained by copolymerisation of the macromonomers A and B in the presence of the monomers NBH and/or NBD.

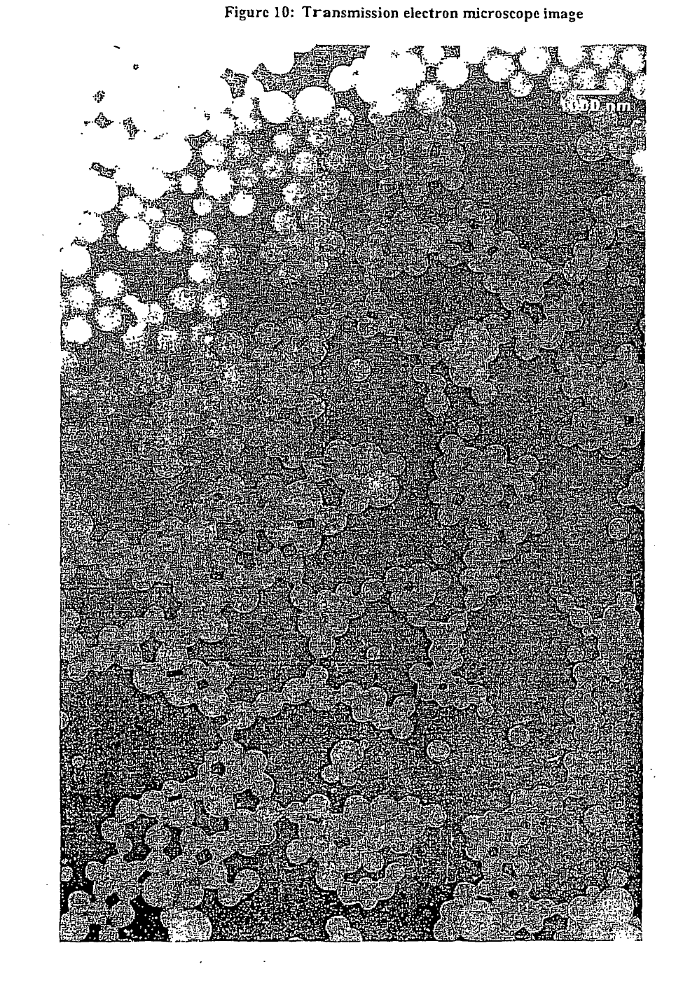

FIG. 11: Size and size distribution of the spherical particles obtained by copolymerisation of the macromonomers A and B in the presence of the monomers NBH and/or NBD, by dynamic diffusion of light.

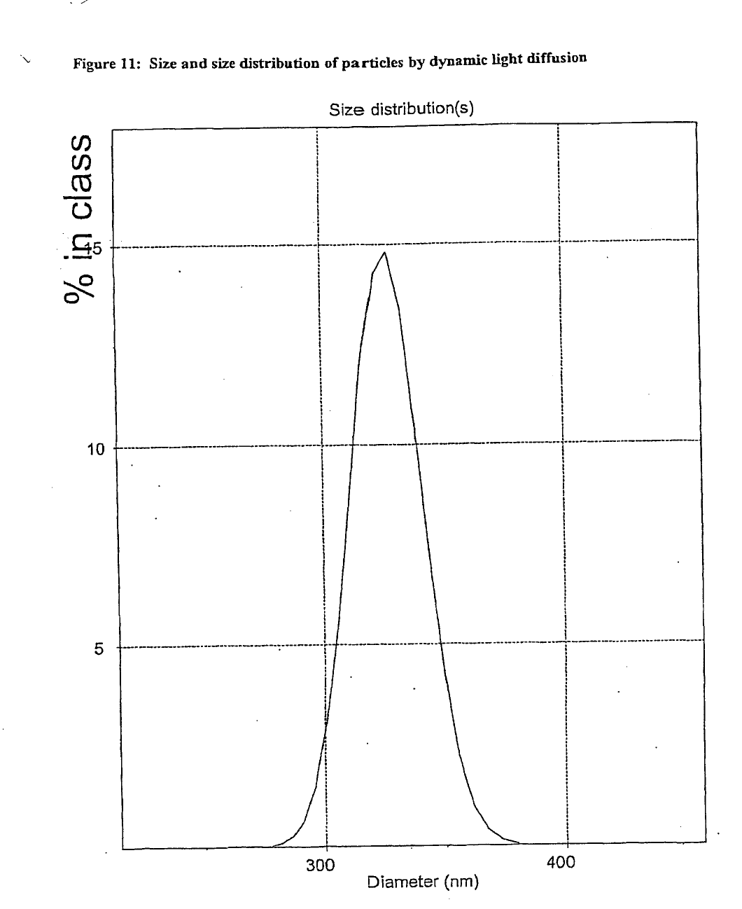

FIG. 12: Steric exclusion chromatography of the spherical particles obtained by copolymerisation of the macromonomers A and B in the presence of the monomers NBH and/or NBD in THF.

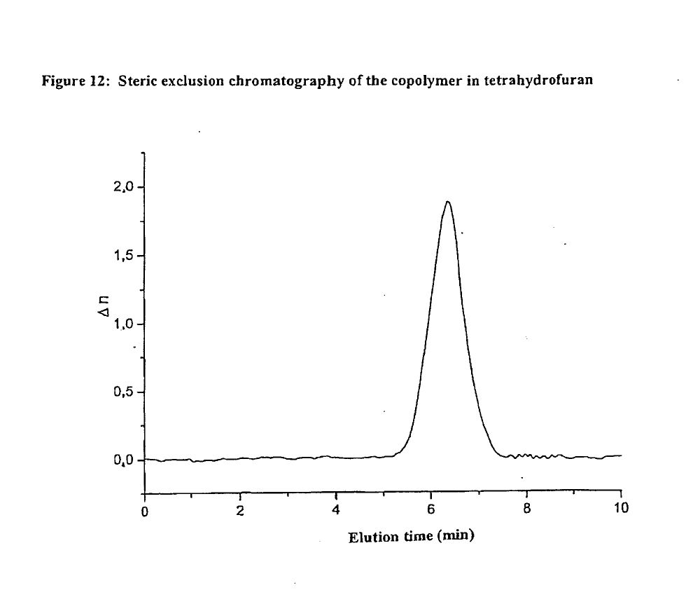

FIG. 13: Representation of a spherical particle according to the invention in which the active ingredient is trapped inside the particle and bonded covalently thereto.

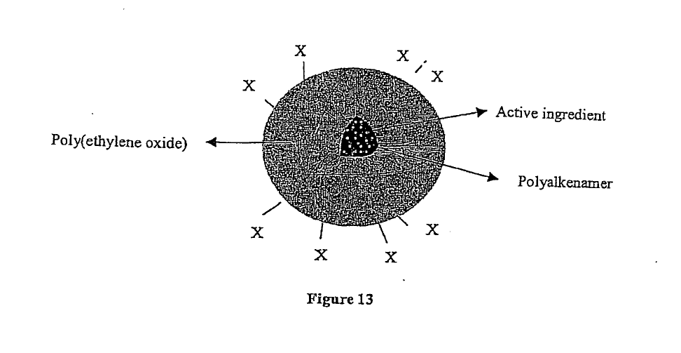

FIG. 14: Illustration of the destabilisation of a spherical particle according to the invention (or latex) and salting out of the medicament.

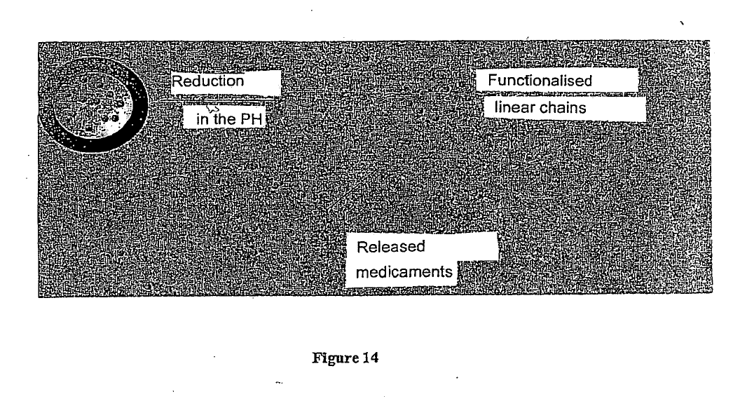

FIG. 15: Observation by scanning electron microscopy of hydroxyapatite.

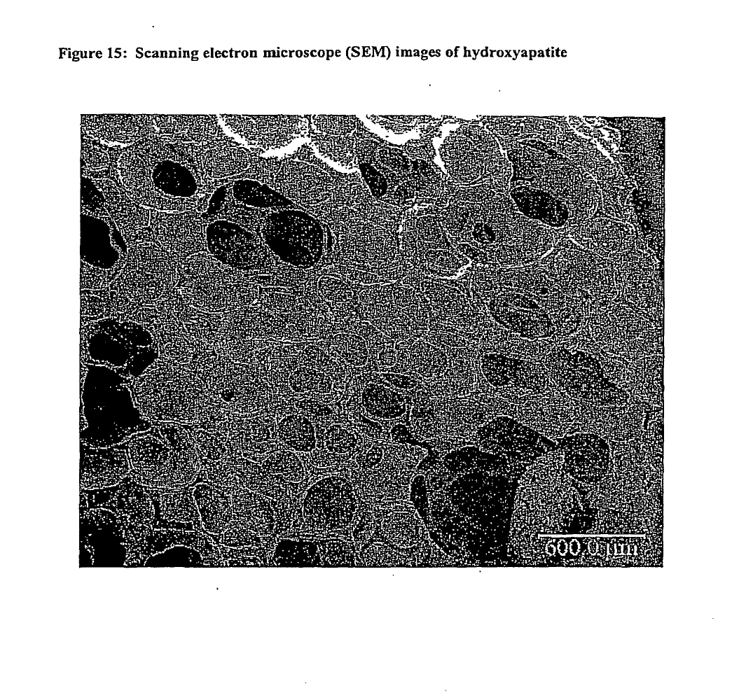

FIG. 16: Observation by scanning electron microscopy of hydroxyapatite+APTES.

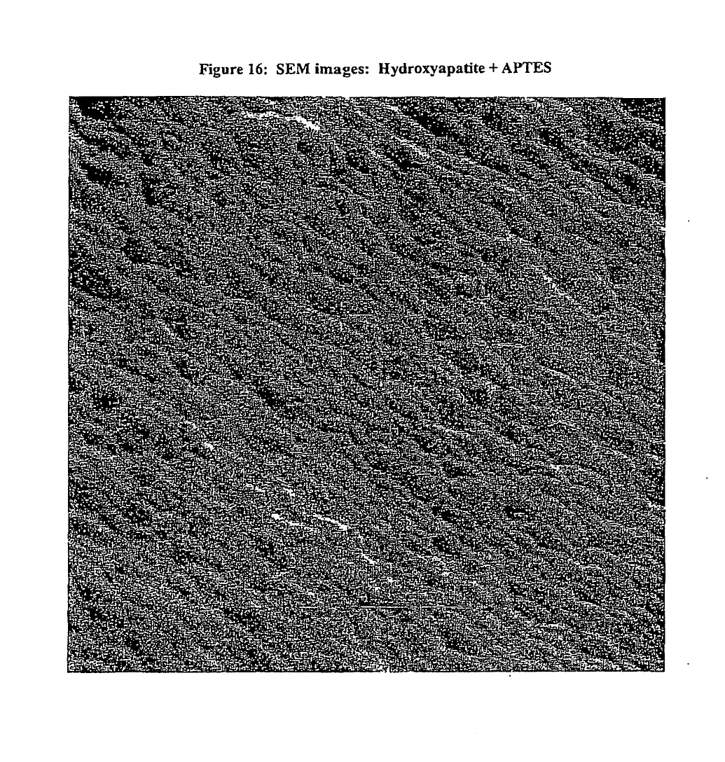

FIG. 17: Observation by scanning electron microscopy of hydroxyapatite+APTES+nanoparticles.

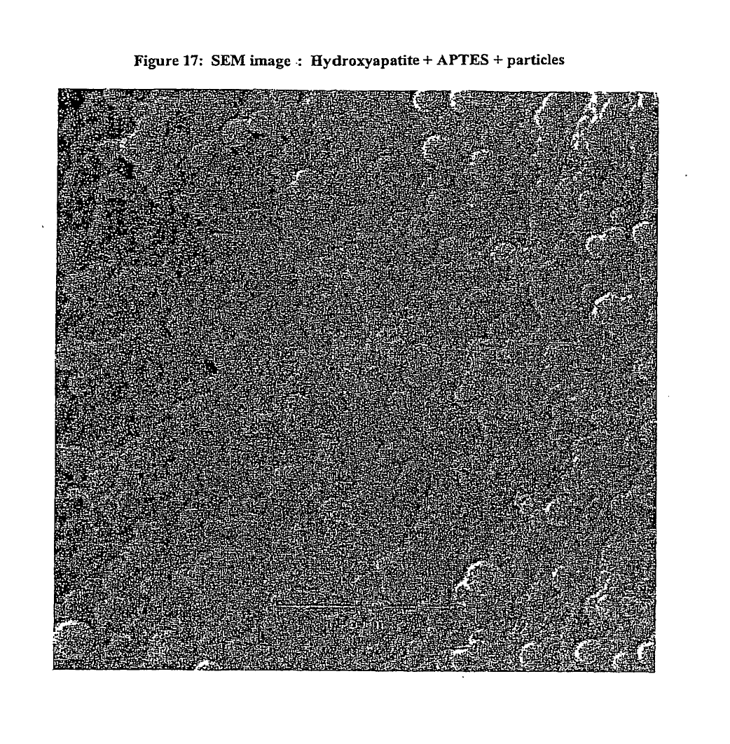

FIG. 18: Observation by scanning electron microscopy of hydroxyapatite+APTES+nanoparticles functionalised after extraction.

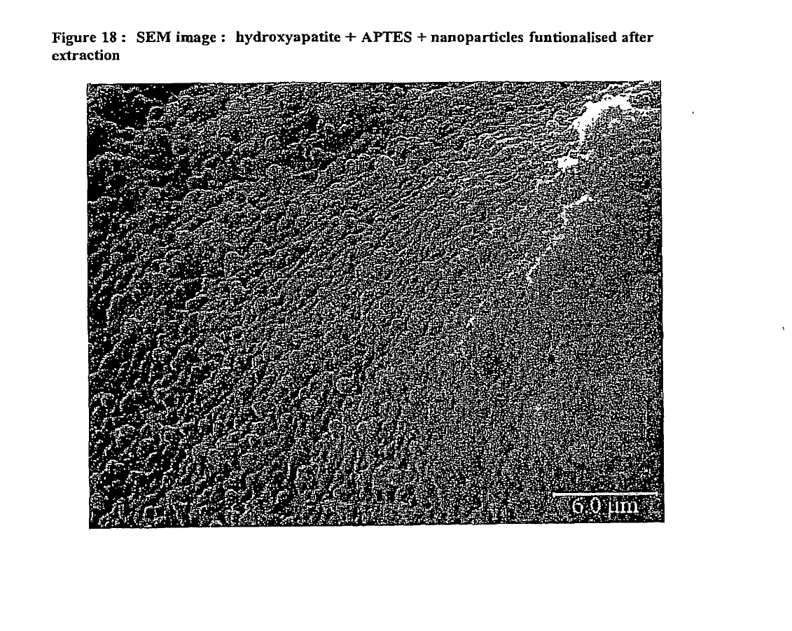

FIG. 19: Observation by scanning electron microscopy of hydroxyapatite+APTES+nanoparticles not functionalised after extraction.

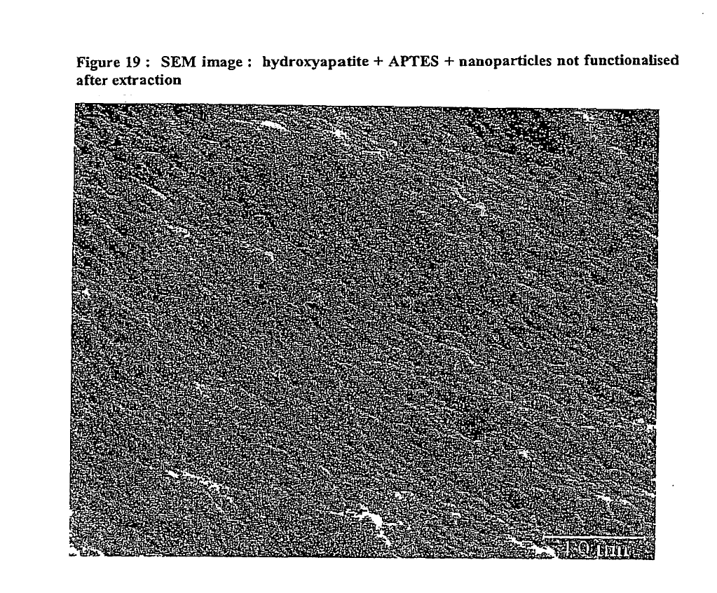

FIG. 20(a, b): XPS spectra of C1 for the materials “A” and “B” respectively.

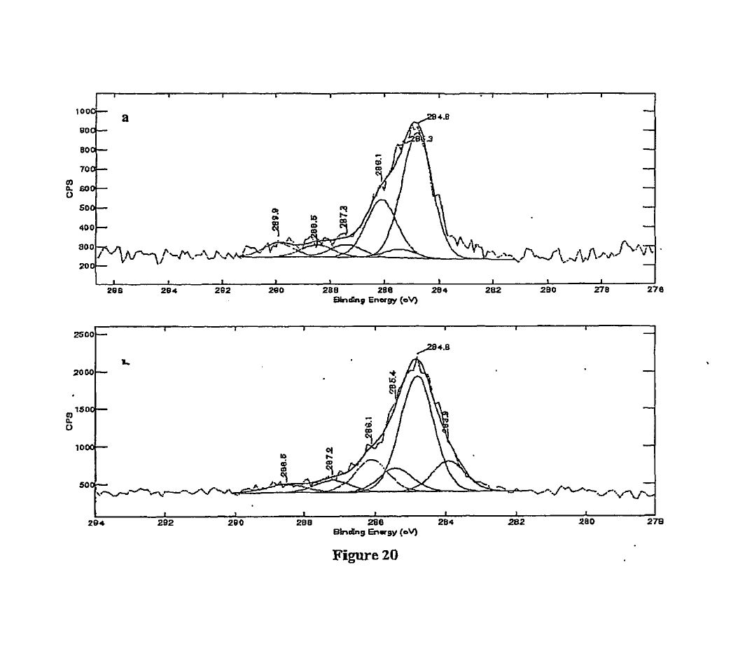

FIG. 21: XPS spectra of N1 for the surfaces of material B.

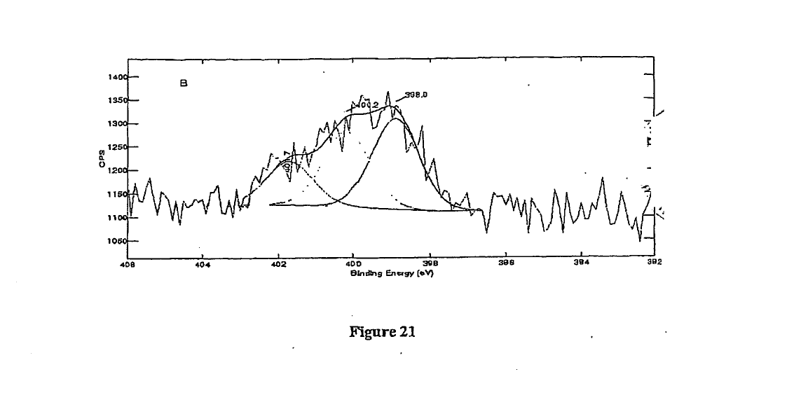

1-18. (canceled)

19. Biomaterials comprising a support material which has covalently bonded on its surface spherical particles having a diameter between 10 nm and 100 μm, said particles being formed by polymer chains containing about 30 to 10000 monomer units, identical or different, derived from the polymerisation of monocyclic alkenes in which the number of carbon atoms constituting the ring is of about 4 to 12 or polycyclic alkenes in which the total number of carbon atoms constituting the rings is of about 6 to 20, the said monomer units being such that:
at least approximately 0.5% of them are substituted by a chain R comprising an ethylene polyoxide of formula (A) optionally covalently bonded to the said monomer units via a hydrolysable bridge
—(CH₂—CH₂—O)ₙ—X (A)
wherein n represents an integer from about 50 to 340, especially from 70 to 200, and X represents an alkyl or alkoxy chain with about 1 to 10 carbon atoms, comprising a reactive function of the OH, halogen, NH₂, C(O)X₁ type in which X₁ represents a hydrogen atom, a halogen atom, an OR′ or NHR′ group wherein R′ represents a hydrogen atom or a hydrocarbon chain with approximately 1 to 10 carbon atoms, substituted or unsubstituted, the said reactive function being capable of bonding to a reactive function situated on the said support material in order to ensure the covalent bonding between the said material and the said particles,
and at least approximately 0.5% of them are substituted by a chain R comprising an ethylene polyoxide of the aforementioned formula (A) in which the said reactive function is engaged in a bond with an active ingredient, or a biological molecule such as a protein, the said chains R being bonded covalently to the said monomers.

20. The biomaterials of claim 1, characterised in that the monomer units are derived from the polymerisation of monocyclic alkenes and are of the following formula (Z1)
═[CH—R₁—CH]═ (Z1)
wherein R₁ represents a hydrocarbon chain with 2 to 10 carbon atoms, saturated or unsaturated, the said monomers being optionally substituted by a chain R, or directly by a group X.

21. The biomaterials of claim 19, characterised in that the monocyclic alkenes from which the monomer units are derived are:
cyclobutene leading to a polymer comprising monomer units of formula (Z1a) below: 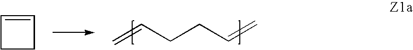
cyclopentene leading to a polymer comprising monomer units of formula (Z1b) below: 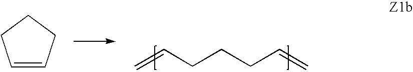
cyclopentadiene leading to a polymer comprising monomer units of formula (Z1c) below: 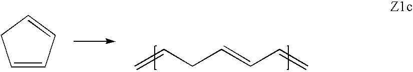
cyclohexene leading to a polymer comprising monomer units of formula (Z1d) below: 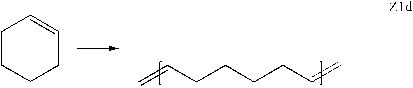
cyclohexadiene leading to a polymer comprising monomer units of formula (Z1e) below: 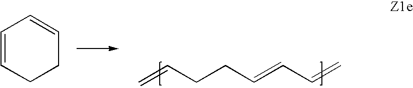
cycloheptene leading to a polymer comprising monomer units of formula (Z1f) below: 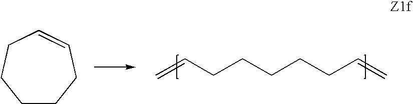
cyclooctene leading to a polymer comprising monomer units of formula (Z1h) below: 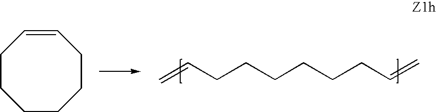
cyclooctapolyene, especially cycloocta-1,5-diene, leading to a polymer comprising monomer units of formula (Z1i) below: 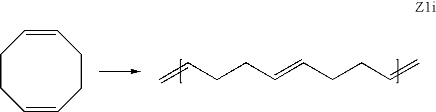
cyclononene leading to a polymer comprising monomer units of formula (Z1j) below: 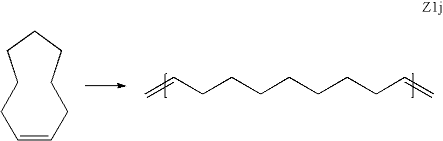
cyclononadiene leading to a polymer comprising monomer units of formula (Z1k) below: 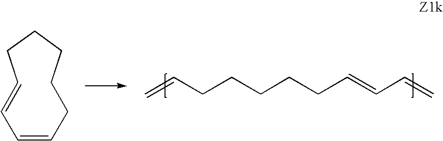
cyclodecene leading to a polymer comprising monomer units of formula (Z1l) below: 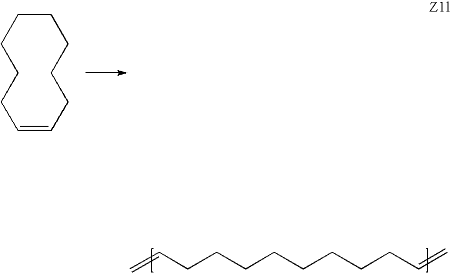
cyclodeca-1,5-diene leading to a polymer comprising monomer units of formula (Z1m) below: 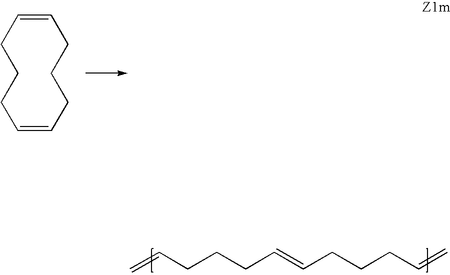
cyclododecene leading to a polymer comprising monomer units of formula (Z1n) below: 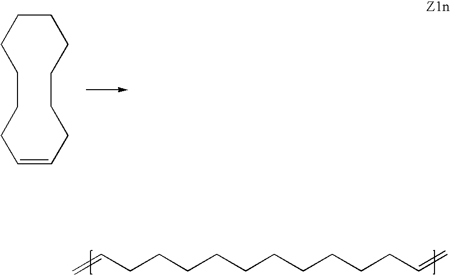
or also 2,3,4,5-tetrahydrooxepin-2-yl acetate, cyclopentadecene, paracyclophane, ferrocenophane.

22. The biomaterials of claim 19, characterised in that the monomer units are derived from the polymerisation of polycyclic alkenes and are:
of formula (Z2) below:
═[CH—R₂—CH]═ (Z2)
wherein R₂ represents :
a ring of formula 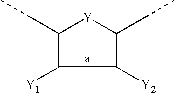
wherein:
Y represents —CH₂—, or a heteroatom, or a —CHR— group, or a —CHX— group, R and X being as previously,
Y₁ and Y₂ independently of one another represent H, or a chain R, or a group X, as mentioned above, or form in association with the carbon atoms bearing them a ring with 4 to 8 carbon atoms, this ring being optionally substituted by a chain R or a group X as mentioned above,
a represents a single or double bond,
or a ring of formula 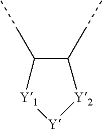
wherein:
Y′ represents —CH₂—, or a heteroatom, or a —CHR— group, or a —CHX— group, R and X being as defined above,
Y′₁ and Y′₂ independently of one another represent —CH₂—, or a —C(O) group, of a —COR group, or a —C—OX group, R and X being as defined above,
of formula (Z3) below: 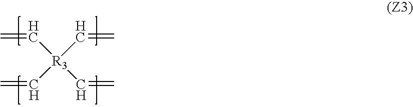
wherein R₃ represents:
a ring of formula 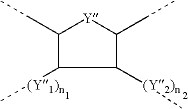
wherein:
n₁ and n₂ independently of one another represent 0 or 1,
Y″ represents —CH₂—, or a —CHR— group, or a —CHX— group, R and X being as defined above,
Y″₁ and Y″₂ independently of one another represent a hydrocarbon chain with 0 to 10 carbon atoms,
or a ring of formula 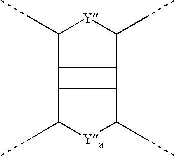
in which Y″ and Y″a independently of one another represent —CH₂—, or a —CHR— group, or a —CHX— group, R and X being as defined above,
or a ring of formula 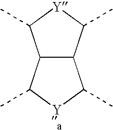
in which Y″ and Y″a independently of one another represent —CH₂—, or a —CHR— group, or a —CHX— group, R and X being as defined above.

23. The biomaterials of claim 19, wherein the polycyclic alkenes from which the monomer units are derived are:
monomers containing a cyclobutene ring leading to a polymer comprising monomer units of formula (Z2a) below: 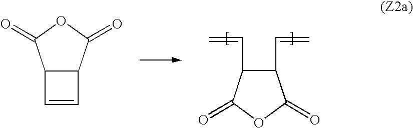
monomers containing a cyclopentene ring leading to a polymer comprising monomer units of formula (Z2b) below: 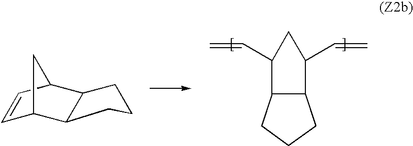
(bicyclo[2.2.1]hept-2-ene)norbornene leading to a polymer comprising monomer units of formula (Z2c) below: 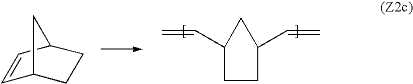
norbornadiene leading to a polymer comprising monomer units of formula (Z2d) below: 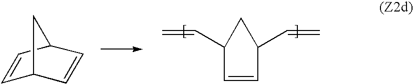
7-oxanorbornene leading to a polymer comprising monomer units of formula (Z2e) below: 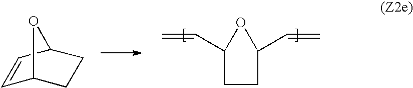
7-oxanorbornadiene leading to a polymer comprising monomer units of formula (Z2f) below: 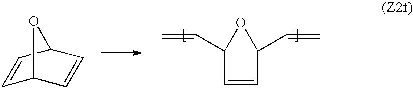
the dimer of norbornadiene leading to a polymer comprising monomer units of formula (Z3a) below: 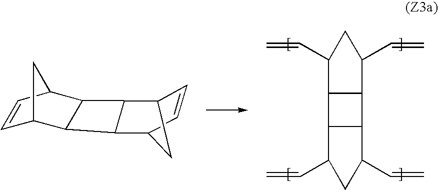
dicyclopentadiene leading to a polymer comprising monomer units of formula (Z3b) below: 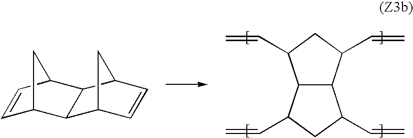
tetracyclododecadiene leading to a polymer comprising monomer units of formula (Z3c) below: 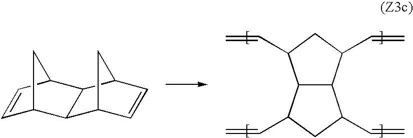
or bicyclo[5.1.0]oct-2-ene, bicyclo[6.1.0]non-4-ene.

24. The biomaterials of claim 19, wherein the monocyclic or polycyclic alkenes from which the monomer units are derived are:
norbornene(bicyclo[2.2.1]hept-2-ene) leading to a polymer comprising monomer units of formula (Z2c),
tetracyclododecadiene leading to a polymer comprising monomer units of formula (Z3c),
dicyclopentadiene leading to a polymer comprising monomer units of formula (Z3b),
the dimer of norbornadiene leading to a polymer comprising monomer units of formula (Z3a),
cycloocta-1,5-diene leading to a polymer comprising monomer units of formula (Z1i).

25. Biomaterials of claim 19, wherein the spherical particles comprise:
between about 0.5% up to 100% of monomer units substituted by a R chain as defined above, said R chain being identical for these monomers, and comprising a reactive function capable of bonding to a reactive function situated on the said support material in order to ensure the covalent bond between the said material and the said particles,
and between about 0.5% and 99.5% of monomer units substituted by a chain R as defined above, the said chain R of these monomers being identical for these monomers, in which the said reactive function is engaged in a bond with an active ingredient, or a biological molecule such as a protein,
and/or between about 0.5% and 99.5% of monomer units directly substituted by a group X as defined above, this group X of these monomers being identical to or different from the group X of the chain R of the preceding monomers,
and/or between about 1% and 99.5% of unsubstituted monomer units,
the total of the percentages of the different monomers mentioned above being 100%.

26. The biomaterials of claim 19, wherein the chain or chains R substituting the monomers are represented by the formula
—CH₂—O—(CH₂—CH₂—O)ₙ—CH₂—CH₂—O—X
in which n is as defined above, and X represents H, —CH₂—COOH, —CH₂—COCl, —CH₂—COY, Y representing an active ingredient, or a biological molecule such as a protein.

27. The biomaterials of claim 19, wherein said chain or chains R comprise an ethylene polyoxide of formula (A) bonded covalently to the said monomer units by a hydrolysable bridge chosen from amongst the chain formations having approximately 1 to 10 units of ε-caprolactone, or —OC(O)—, —C(O)OC(O)—, —C(O)—NH— functions.

28. The biomaterials of claim 19, wherein said chain or chains R comprise an ethylene polyoxide of formula (A) covalently bonded to a hydrolysable bridge chosen from amongst the chain formations having approximately 1 to 10 units of ε-caprolactone are represented by the formula
—CH₂—(O—CO—(CH₂)₅)ₜ—O—CO—(CH₂)₅—O—CO—(CH₂)₂—CO—O—(CH₂—CH₂—O)ₙ—(CH₂)₂—O—X
in which t represents a whole number between 1 and 10, and X represents H, —CH₂—COOH, —CH₂—COCl or —CH₂—COY, Y representing an active ingredient, or a biological molecule such as a protein.

29. The biomaterials of claim 19, wherein said support material is chosen from
metals, such as titanium,
metal alloys, in particular alloys with or without shape memory such as Ni—Ti alloys,
polymers, such as polyethylene terephthalate (PET), polytetrafluoroethylene (PTFE), polyvinylidine fluoride (PVDF), polyether etherketone (PEEK),
copolymers, such as the copolymer ethylene vinyl acetate (EVA), the copolymer vinylidene fluoride-hexafluoropropylene P(VDF-HFP), poly(lactic acid)-co-poly(glycolic acid) (PLA-PGA),
ceramics, such as hydroxyapatites, or compounds of hydroxyapatites and tricalcium phosphate in varied proportions, in particular in the proportions 50/50.

30. The biomaterials of claim 19, wherein said reactive function situated on the support material in order to ensure the covalent bond between the said material and the said particles by reacting with the reactive function of these latter is of the type of OH, halogen, NH₂, C(O)X′₁ wherein X′₁ represents a hydrogen atom, a halogen atom, an OR″ or NHR″ group, wherein R″ represents a hydrogen atom or a hydrocarbon chain with about 1 to 10 carbon atoms, substituted or unsubstituted, in order to form a bond of the —O—C(O)—, —NH—C(O)—, —C(O)—NH—, —C(O)0- or —C(OC)₂ type with the reactive function of said particles.

31. The biomaterials of claim 19, wherein said reactive function of the support material is situated on an alkyl chain having approximately 1 to 10 carbon atoms grafted on said material, substituted or unsubstituted, and optionally comprising one or several heteroatoms, in particular O, and Si, in the said chain.

32. The biomaterials of claim 19, wherein:
the reactive function of the material is an NH₂ function situated on an aminopropyltriethoxysilane molecule grafted on the material according to the following formulae: 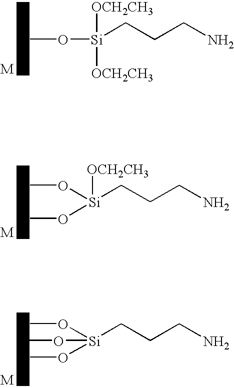
wherein M represents a metal oxide or a ceramic such as hydroxyapatite or any other polymer having OH sites on its surface (naturally or due to prefunctionalisation),
the reactive function of the material is an NH₂ function situated on a surface prefunctionalised by acrylic acid which is coupled to a bifunctional spacer arm such as bNH₂PEG (O,O′-bis-(2-aminopropyl)-polyethylene glycol 500 (this prefunctionalisation is described in the article Nucl. Instr. And Meth. in Phys. Res. B 151 1999 255-262).

33. The biomaterials of claim 19, wherein the active ingredient is chosen from the molecules used in therapy, cosmetics, perfumery, or for surface coatings, such as paints and antifouling coatings.

34. The biomaterials of claim 19, wherein the active ingredient is a medicament used in therapy chosen in particular from those in the following therapeutic categories: antibiotics, antiinflammatories, antimitotics, hormones, growth factors.

35. The use of biomaterials of claim 19 for the preparation of implantable medical devices, in particular in the form of implants, prostheses, stents or cements, in particular in vascular, endovascular or bone surgery.

36. Implants, prostheses, vascular stents or cements comprising biomaterials according to claim 19.
